(19) United States
(12) Patent Application Publication
(10) Pub. No.: US 2022/0387433 A1
(43) Pub. Date: Dec. 8, 2022

(54) TYPE V PHOSPHODIESTERASE INHIBITOR COMPOSITIONS, METHODS OF MAKING THEM AND METHODS OF USING THEM IN PREVENTING OR TREATING ELEVATED PULMONARY VASCULAR PRESSURE OR PULMONARY HEMORRHAGES

(71) Applicant: American Regent, Inc., Shirley, NY (US)

(72) Inventors: Richard Lawrence, Southold, NY (US); Kenneth C. Thompson, Hillsborough, NJ (US); Sanjay Kumar Singh, South Setauket, NY (US)

(73) Assignee: American Regent, Inc., Shirley, NY (US)

(21) Appl. No.: 17/775,794

(22) PCT Filed: Nov. 10, 2020
(86) PCT No.: PCT/US20/59861

§ 371 Date: May 10, 2022

Related U.S. Application Data
(60) Provisional application No. 62/934,308, filed on Nov. 12, 2019.

(51) Int. Cl. A61K 31/519; A61K 9/00; A61K 31/517; A61K 31/506; A61K 31/13; A61K 31/53; A61K 38/00

(52) U.S. Cl. CPC A61K 31/519; A61K 9/0019; A61K 31/517; A61K 31/506; A61K 31/13; A61K 31/53; A61K 38/005

(57) ABSTRACT

Methods of preventing or treating elevated pulmonary vascular pressure or exercise-induced pulmonary hemorrhage in mammals are provided, the methods comprise administering compositions comprising type V phosphodiesterase inhibitors and an organic base (e.g., meglumine) to the mammal. Compositions, kits and methods of making type V phosphodiesterase inhibitor are also provided. In one embodiment, the composition comprises E-4021, which is sodium 1-[6-chloro-4-(3,4-methylenedioxybenzyl)-aminoquinazolin-2-yl]piperidine-4-carboxylate sesquihydrate and meglumine.

### BACKGROUND

Elevated pulmonary vascular pressure is a type of high blood pressure that affects the arteries in the lungs where the pulmonary pressure is elevated beyond normal pressure. Elevated pulmonary vascular pressure, particularly after exercise, can lead to a severe condition known as exercise-induced pulmonary hemorrhage in mammals including humans.

Exercise-induced pulmonary hemorrhage (EIPH), also known as “bleeding” or a “bleeding attack,” refers to the accumulation of blood in the airways of the lung in association with exercise. EIPH is common in horses undertaking intense exercise, but it has also been reported in human athletes, racing camels, racing greyhounds and humans with diseases such as left heart failure.

In thoroughbred racehorses, after the race and the excitement of the race is over, unfortunately most of the racehorses develop some form of EIPH. Often times, the EIPH can be severe and the racehorse has to be euthanized.

Mammals with EIPH may be referred to as “bleeders” or as having “broken a blood vessel.” Often times, EIPH is not always apparent and can be detected by a tracheobronchoscopic assessment examination of the airways performed following exercise. However, some mammals may show bleeding at the nostrils after exercise, known as epistaxis.

A number of treatments have been used or suggested for EIPH, including resting, anti-inflammatories (e.g., corticosteroids), bronchodilators, anti-hypertensive agents (including nitric oxide donors and phosphodiesterase inhibitors), conjugated estrogens (e.g., Premarin®), antifibrinolytics (e.g., aminocaproic acid and tranexamic acid), snake venom, aspirin, vitamin K, bioflavonoids, diuretics (e.g., furosemide, known as Lasix® or Salix®), nasal strips, and omega-3 fatty acids.

Although furosemide is a common treatment used in racehorses, it is believed to be ineffective in a large number of subjects. Furosemide may also improve racing times in horses both with and without EIPH, possibly due to a lowering of body weight as a consequence of its potent diuretic action. The use of furosemide in competing horses is therefore prohibited in some countries, and it is regarded as a banned substance by the International Olympic Committee. Moreover, chronic usage of furosemide can lead to hypokalemia and hypomagnesemia. Finally, the diuretic effects of furosemide can lead to dehydration, which can be detrimental to the health of subjects engaging in athletic activities.

Therefore, there is a need for new methods and compositions for treating or preventing elevated pulmonary vascular pressure or EIPH, with improved efficacy, duration of action, stability, and fewer side-effects.

### SUMMARY

New methods and compositions for treating or preventing elevated pulmonary vascular pressure or EIPH, with improved efficacy, duration of action, stability, and fewer side-effects are provided.

In one embodiment, there is a method of preventing or treating elevated pulmonary vascular pressure or exercise-induced pulmonary hemorrhage in a mammal in need thereof, the method comprising administering a composition comprising a type V phosphodiesterase inhibitor, an alcohol and water to the mammal.

In another embodiment, there is a method of preventing or treating elevated pulmonary vascular pressure or exercise-induced pulmonary hemorrhage in a mammal in need thereof, the method comprising administering a composition comprising a type V phosphodiesterase inhibitor and an organic base or amino sugar to the mammal.

The composition can be administered systemically or locally. For example, the composition can be administered intravenously to a mammal such as, for example, a horse, a dog, a camel, a monkey, a cat, a pig, a cow, a goat, a llama, a sheep, a mouse, a rat, a rabbit, or a human.

In one exemplary embodiment, the type V phosphodiesterase inhibitor comprises E-4021, which is sodium 1-[6-chloro-4-(3,4-methylenedioxybenzyl)-aminoquinazolin-2-yl]piperidine-4-carboxylate sesquihydrate and the organic base or amino sugar comprises meglumine. In some embodiments, the composition can have less toxicity, such as for example, blood in the urine and/or renal toxicity. In some embodiments, the composition has improved stability and an extended duration of action more than 24 hours after dose administration.

In another embodiment, there is a method of making a composition for preventing or treating elevated pulmonary vascular pressure or exercise-induced pulmonary hemorrhage in a mammal in need thereof, the method comprising adding an organic base (e.g., meglumine) to a solution of a type V phosphodiesterase inhibitor to form the composition.

In yet another embodiment, there is an aqueous composition for preventing or treating exercise-induced elevated pulmonary vascular pressure or pulmonary hemorrhage in a mammal, the aqueous composition comprising a type V phosphodiesterase inhibitor, an organic base (e.g., meglumine) and water.

In still yet another embodiment, there is a kit for the treatment or prevention of elevated pulmonary vascular pressure or exercise-induced pulmonary hemorrhage in a subject in need thereof, the kit comprising a composition comprising a type V phosphodiesterase inhibitor, an amino sugar such as meglumine and water.

In some embodiments, there is a method of increasing the duration of action of a type V phosphodiesterase inhibitor, the method comprising adding an organic base to the type V phosphodiesterase inhibitor to form an aqueous injectable solution having a pH between about 7.1 to about 12.

In some embodiments, there is a composition comprising sodium 1-[6-chloro-4-(3,4-methylenedioxybenzyl)-aminoquinazolin-2-yl]piperidine-4-carboxylate sesquihydrate, meglumine, and alcohol.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE FIGURES

In part, other aspects, features, benefits and advantages of the embodiments will be apparent with regard to the following description, appended claims and accompanying figures.

FIG. 1 is a bar graph illustration of pulmonary arterial pressure (PAP) for horses. Mean±Standard Error (SE) pulmonary artery pressure measured during Phase II (Top) and Phase III (Bottom). Pressure measured at the end of the 2 minute 6 m/s warm-up (WU-2 min), at 1 minute (EX-1 min) and 2 minutes (EX-2 min) of the high speed run at 110% of the speed required to elicit VO₂ₘₐₓ, and at the end of the 4 m/s cool down period (REC-2 min).

FIG. 2 is a comparative bar graph illustration of pulmonary arterial pressure for horses involved in a 1^{st} Trial and a 2^{nd} Trial. Mean±SE pulmonary artery pressure measured at 2 minutes (EX-2 min) of the high-speed run at 110% of the speed required to elicit VO₂ₘₐₓ during Phase II (1^{st} Trial) and Phase III (2^{nd} Trial).

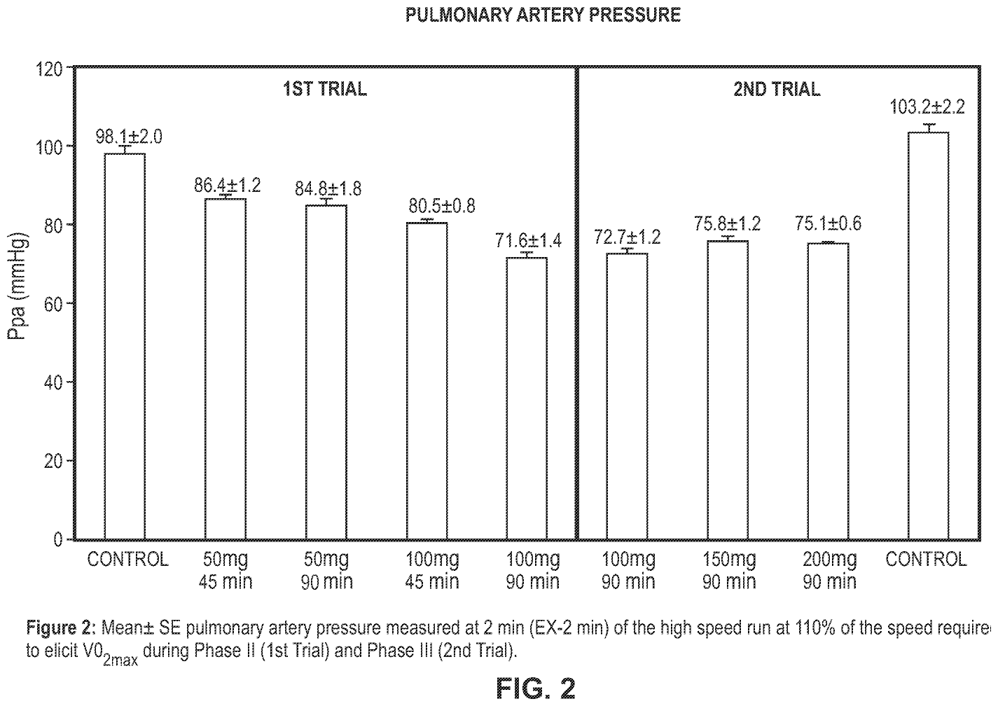

FIG. 3 is a bar graph illustration of oxygen uptake for horses. Mean±SE oxygen uptake (VO₂) measured during Phase II (Top) and Phase III (Bottom) at the end of the 2 minute 6 m/s warm-up (WU-2 min), at 1 minute (EX-1 min) and 2 minutes (EX-2 min) of the high speed run at 110% of the speed required to elicit VO₂ₘₐₓ, and at the end of the 4 m/s cool down period (REC-2 min).

FIG. 4 is a bar graph illustration of plasma lactate for horses. Mean+SE plasma lactate measured during Phase II (Top) and Phase III (Fi) before exercise (PRE-EX), at the end of the 2 minute 6 m/s warm-up (WU-2 min), at 1 minute (EX-1 min) and 2 minutes (EX-2 min) of the high speed run at 110% of the speed required to elicit VO₂ₘₐₓ, and at the end of the 4 m/s cool down period (REC-2 min).

FIG. 5 is a bar graph illustration of plasma glucose concentration for horses. Mean±SE plasma glucose concentration measured during Phase II (Top) and Phase III (Bottom) before exercise (PRE-EX), at the end of the 2 minute 6 m/s warm-up (WU-2 min), at 1 minute (EX-1 min) and 2 minutes (EX-2 min) of the high speed run at 110% of the speed required to elicit VO₂ₘₐₓ, and at the end of the 4 m/s cool down period (REC-2 min).

FIG. 6 is a bar graph illustration of venous partial pressure of oxygen measured in pulmonary artery blood for horses. Mean±SE venous partial pressure of oxygen measured in pulmonary artery blood P_{PA}O₂ during Phase II (Top) and Phase III (Bottom) before exercise (PRE-EX), at the end of the 2 minute 6 m/s warm-up (WU-2 min), at 1 minute (EX-1 min) and 2 minutes (EX-2 min) of the high speed run at 110% VO₂ₘₐₓ, and at the end of the 4 m/s cool down period (REC-2 min).

FIG. 7 is a bar graph illustration of venous pH measured in pulmonary artery blood for horses. Mean±SE venous pH measured in pulmonary artery blood during Phase II (Top) and Phase III (Bottom) before exercise (PRE-EX), at the end of the 2 minute 6 m/s warm-up (WU-2 min), at 1 minute (EX-1 min) and 2 minutes (EX-2 min) of the high speed run at 110% of the speed required to elicit VO₂ₘₐₓ, and at the end of the 4 m/s cool down period (REC-2 min).

FIG. 8 is a bar graph illustration of venous oxygen saturation measured in pulmonary artery blood for horses. Mean±SE venous oxygen saturation measured in pulmonary artery blood during Phase II (Top) and Phase III (Bottom) before exercise (PRE-EX), at the end of the 2 minute 6 m/s warm-up (WU-2 min), at 1 minute (EX-1 min) and 2 minutes (EX-2 min) of the high speed run at 110% of the speed required to elicit VO₂ₘₐₓ, and at the end of the 4 m/s cool down period (REC-2 min).

FIG. 9 is a bar graph illustration of venous partial pressure of carbon dioxide measured in pulmonary artery blood for horses. Mean±SE venous partial pressure of carbon dioxide measured in pulmonary artery blood during Phase II (Top) and Phase III (Bottom) before exercise (PRE-EX), at the end of the 2 minute 6 m/s warm-up (WU-2 min), at 1 minute (EX-1 min) and 2 minutes (EX-2 min) of the high speed run at 110% of the speed necessary to elicit VO₂ₘₐₓ, and at the end of the 4 m/s cool down period (REC-2 min).

FIG. 10 is a bar graph illustration of venous base ecf measured in pulmonary artery blood for horses. Mean±SE venous base ecf measured in pulmonary artery blood during Phase II (Top) and Phase III (Bottom) before exercise (PRE-EX), at the end of the 2 minute 6 m/s warm-up (WU-2 min), at 1 minute (EX-1 min) and 2 minutes (EX-2 min) of the high speed run at 110% of the speed required to elicit VO₂ₘₐₓ, and at the end of the 4 m/s cool down period (REC-2 min).

FIG. 11 is a bar graph illustration of venous hemoglobin content for horses. Mean±SE venous hemoglobin content measured during Phase II (Top) and Phase III (Bottom) before exercise (PRE-EX), at the end of the 2 minute 6 m/s warm-up (WU-2 min), at 1 minute (EX-1 min) and 2 minutes (EX-2 min) of the high speed run at 110% of the speed necessary to elicit VO₂ₘₐₓ, and at the end of the 4 m/s cool down period (REC-2 min).

FIG. 12 is a bar graph illustration of venous packed cell volume for horses. Mean±SE venous packed cell volume measured during Phase II (Top) and Phase III (Bottom) before exercise (PRE-EX), at the end of the 2 minute 6 m/s warm-up (WU-2 min), at 1 minute (EX-1 min) and 2 minutes (EX-2 min) of the high speed run at 110% of the speed necessary to elicit VO₂ₘₐₓ, and at the end of the 4 m/s cool down period (REC-2 min).

FIG. 13 is a bar graph illustration of venous plasma sodium concentration for horses. Mean±SE venous plasma sodium concentration measured during Phase II (Top) and Phase III (Bottom) before exercise (PRE-EX), at the end of the 2 minute 6 m/s warm-up (WU-2 min), at 1 minute (EX-1 min) and 2 minutes (EX-2 min) of the high speed run at 110% necessary to elicit VO₂ₘₐₓ, and at the end of the 4 m/s cool down period (REC-2 min).

FIG. 14 is a bar graph illustration of venous plasma potassium concentration for horses. Mean±SE venous plasma potassium concentration measured during Phase II (Top) and Phase III (Bottom) before exercise (PRE-EX), at the end of the 2 minute 6 m/s warm-up (WU-2 min), at 1 minute (EX-1 min) and 2 minutes (EX-2 min) of the high speed run at 110% of the speed necessary to elicit VO₂ₘₐₓ, and at the end of the 4 m/s cool down period (REC-2 min).

FIG. 15 is a bar graph illustration of venous plasma calcium concentration for horses. Mean±SE venous plasma calcium concentration measured during Phase II (Top) and Phase III (Bottom) before exercise (PRE-EX), at the end of the 2 minute 6 m/s warm-up (WU-2 min), at 1 minute (EX-1 min) and 2 minutes (EX-2 min) of the high speed run at 110% of the speed required to elicit VO₂ₘₐₓ, and at the end of the 4 m/s cool down period (REC-2 min).

FIG. 16 is a bar graph illustration of pulmonary arterial pressure for horses using a single dose of 100 mg PDE5 (15005) injection 90 minutes prior to a Simulated Race Test (SRT).

FIG. 17 is a bar graph illustration of oxygen uptake for horses using a single dose of 100 mg PDE5 (15005) injection 90 minutes prior to SRT.

FIG. 18 is a bar graph illustration of plasma lactate for horses using a single dose of 100 mg PDE5 (15005) injection 90 minutes prior to SRT.

FIG. 19 is a bar graph illustration of venous oxygen saturation measured in pulmonary artery blood for horses using a single dose of 100 mg PDE5 (15005) injection 90 minutes prior to SRT.

FIG. 20 is a bar graph illustration of pulmonary arterial pressure for horses using a single dose of 100 mg PDE5 (16006) at different time points to study the duration of pulmonary artery pressure reduction effect.

FIG. 21 is a bar graph illustration of pulmonary arterial pressure for horses using a single dose of 100 mg PDE5 containing propylene glycol (PPG) and a single dose of 100 mg PDE5 containing meglumine (MEG) (new formulation) respectively at different time points to study the duration of pulmonary artery pressure reduction effect.

FIG. 22 is a bar graph illustration of oxygen uptake for horses using a single dose of 100 mg PDE5 (16006-containing MEG) at different time points.

FIG. 23 is a bar graph illustration of plasma lactate for horses using a single dose of 100 mg PDE5 (16006-containing MEG) at different time points.

FIG. 24 is a bar graph illustration of venous oxygen saturation measured in pulmonary artery blood for horses using a single dose of 100 mg PDE5 (16006-containing MEG) at different time points.

### DETAILED DESCRIPTION

For the purposes of promoting an understanding of the principles of the disclosure, reference will now be made to certain embodiments and specific language will be used to describe the same. It will nevertheless be understood that no limitation of the scope of the disclosure is thereby intended, such alterations and further modifications in the illustrated device, and such further applications of the principles of the disclosure as described herein being contemplated as would normally occur to one skilled in the art to which the disclosure relates.

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present application. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical representations are as precise as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

Additionally, unless defined otherwise or apparent from context, all technical and scientific terms used herein have the same meanings as commonly understood by one of ordinary skill in the art to which this disclosure belongs.

Unless explicitly stated or apparent from context, the following terms are phrases have the definitions provided below:

### Definitions

It is noted that, as used in this specification and the appended claims, the singular forms “a,” “an,” and “the,” include plural referents unless expressly and unequivocally limited to one referent. Thus, for example, reference to “a dose” includes one, two, three or more doses.

The term “mammal” refers to organisms from the taxonomy class “mammalian,” including but not limited to humans, other primates such as chimpanzees, apes, orangutans and monkeys, rats, mice, cats, dogs, cows, horses, camels, pigs, goats, llamas, sheep, or rabbits. In certain embodiments, the mammal is a horse. In some embodiments, the mammal is a human. In some embodiments, the mammal has been diagnosed with elevated pulmonary vascular pressure or EIPH. In some embodiments, the mammal is suspected to have or will have elevated pulmonary vascular pressure or EIPH. In some embodiments, the mammal is at risk for developing elevated pulmonary vascular pressure or EIPH. In some embodiments, a mammal with elevated pulmonary vascular pressure or EIPH is identified by epistaxis. In some embodiments, the mammal is identified by tracheobronchoscopic assessment, bronchoalveolar lavage, biopsy, radiograph, and/or pulmonary scintigraphy. In some embodiments, a mammal at risk for developing elevated pulmonary vascular pressure or EIPH is identified by a history of an elevated pulmonary blood pressure or EIPH.

“Elevated pulmonary vascular pressure” is a condition that includes an increase in the pulmonary vascular pressure of at least 10 mm Hg or more than the normal pulmonary vascular pressure in the mammal. This increase in the pulmonary vascular pressure may occur, in some embodiments, with or without exercise. In some embodiments, pulmonary vascular pressure greater than 90 mm Hg during exercise is considered elevated pulmonary vascular pressure. Elevated pulmonary vascular pressure can cause lung injury and lead to EIPH in the mammal.

As used herein, the term “treatment” or “treating” is defined as the application or administration of a composition useful within the current application (alone or in combination with another agent), to a mammal, who has a physiological condition contemplated herein, a symptom of or the potential to develop a physiological condition contemplated herein, with the purpose to prevent, cure, heal, alleviate, relieve, alter, remedy, ameliorate, improve or affect a physiological condition contemplated herein, the symptoms of or the potential to develop a physiological condition contemplated herein. Similar considerations apply to improving the physiological functions or parameters contemplated within the current application. As used herein, the term “treat” means reducing the frequency with which symptoms are or may be experienced by a mammal or administering a compound to reduce the severity with which symptoms are or may be experienced.

As used herein, “alleviating a condition,” means reducing the severity of the symptom of the condition.

As used herein, a “prophylactic” treatment is a treatment administered to a subject who does not exhibit signs of a condition or exhibits only early signs of the condition for the purpose of decreasing the risk of developing pathology associated with the condition.

As used herein, the term “preventing” or “prevention” means no condition development if none had occurred, or no further condition development if there had already been development of the condition. Also considered is the ability of one to prevent some or all of the symptoms associated with the condition.

As used herein, the term “effective amount” of a compound or composition refers to the amount of the compound or composition that is sufficient to provide a beneficial effect to the subject to which the compound or composition is administered.

As used herein, the term “acceptable” refers to a material, such as a carrier or diluent, which does not abrogate the biological activity or properties of the compound or composition, and is relatively non-toxic, i.e., the material may be administered to an individual without causing undesirable biological effects or interacting in a deleterious manner with any of the components of the composition in which it is contained.

As used herein, the language “acceptable salt” refers to a salt of the administered compounds prepared from acceptable non-toxic acids, including inorganic acids, organic acids, solvates, hydrates, or clathrates thereof. The type V phosphodiesterase inhibitors of the present application can be in the composition as a pharmaceutically acceptable salt.

As used herein, the term “composition” refers to a mixture of at least one compound useful within the current application with other chemical components, such as carriers, stabilizers, diluents, dispersing agents, suspending agents, thickening agents, and/or excipients. The composition facilitates administration of the compound to the mammal. Compositions refer to a mixture that usually contains a carrier, such as a pharmaceutically acceptable carrier or excipient, which is suitable for administration into a subject for therapeutic, diagnostic, or prophylactic purposes.

Multiple techniques of administering a composition exist in the art including, but not limited to, administration by intravenous (e.g., intravenous push, intravenous infusion, etc.), intramuscular, subcutaneous, intraperitoneal, intraarterial, inhalation, intradermal, oral, topical or ophthalmic administration.

The term “solution” refers to a homogeneous liquid preparation that contains one or more chemical substances dissolved (e.g., molecularly dispersed), in a suitable solvent or mixture of mutually miscible solvents. Typically, solutions are mixtures with particle sizes of less than 10⁻⁷ cm.

The term “suspension” refers to a two-phase system with uniform dispersion of finely divided solid particles in a continuous phase of liquid in which the particles have minimum solubility and a particle size greater than 10⁻⁵ cm. Here in suspensions, the finely divided solid particles are called as dispersed phase or external phase or discontinuous phase and the phase in which they are dispersed is called as dispersion medium or internal phase or continuous phase.

The duration of action of a drug is the length of time that particular drug is effective. Duration of action is a function of several parameters including plasma half-life, the time to equilibrate between plasma and target compartments, and the off rate of the drug from its biological target.

Reference will now be made in detail to certain embodiments of the disclosure. The disclosure is intended to cover all alternatives, modifications, and equivalents that may be included within the disclosure as defined by the appended claims.

The headings below are not meant to limit the disclosure in any way; embodiments under any one heading may be used in conjunction with embodiments under any other heading.

### Type V Phosphodiesterase Inhibitors

New methods and compositions for treating or preventing elevated pulmonary vascular pressure or EIPH, with improved efficacy, duration of action, stability, and fewer side-effects are provided. Elevated pulmonary vascular pressure is a condition that includes high blood pressure that affects the arteries in the lungs. Elevated pulmonary vascular pressure can lead to EIPH, which refers to the presence of blood in the airways of the lung, which is often associated with exercise. For example, in an exercising horse, a pulmonary arterial pressure threshold exists above which hemorrhage occurs, and that pressure is often exceeded during high speed sprint exercise. Exercise-induced pulmonary hemorrhage (EIPH) can be characterized by blood in the airways after strenuous exercise and results from stress failure of the pulmonary capillaries.

Type V phosphodiesterase inhibitor can be used to treat elevated pulmonary vascular and EIPH as described in U.S. Pat. No. 8,217,049 assigned to American Regent, Inc. The entire disclosure of this patent is herein incorporated by reference.

The type V phosphodiesterase inhibitor compositions of the present application also contain one or more organic bases. Type V phosphodiesterase inhibitors suitable for use in the present application block the action of cGMP-specific phosphodiesterase type 5 (PDE5) on cyclic GMP. The type V phosphodiesterase inhibitors act as pulmonary vasodilators.

Suitable type V phosphodiesterase inhibitors for use in the present application, include but are not limited to, sildenafil, avanafil, iodenafil, mirodenafil, tadalafil, vardenafil, udenafil, zaprinast, icariin and its synthetic derivatives, benzamidenafil, dasantafil, dipyridamole, tadalafil, E4021 (sodium 1-[6-chloro-4-(3,4-methylenedioxybenzyl)-aminoquinazolin-2-yl]piperidine-4-carboxylate sesquihydrate) (available from Eisai Co., Ltd., Tokyo, Japan), E4010, which is 4-(3-chloro-4-methoxybenzyl)amino-1-(4-hydroxypiperidino)-6-phthalazinecarbonitrile monohydrochloride, DMPPO (1,3-dimethyl-6-(2-propoxy-5-methanesulfonylamidophenyl)pyrazol[3,4d]-pyr-imidin-4-(5H)-one) or a combination thereof.

The type V phosphodiesterase inhibitors may be in a pharmaceutically acceptable salt form, which refers to a salt of the administered compounds prepared from acceptable non-toxic acids, including inorganic acids, organic acids, solvates, hydrates, or clathrates thereof. The type V phosphodiesterase inhibitors of the present application can be in the composition as a pharmaceutically acceptable salt.

In one embodiment, the composition comprises the type V phosphodiesterase inhibitor E4021 (sodium 1-[6-chloro-4-(3,4-methylenedioxybenzyl)-aminoquinazolin-2-yl]piperidine-4-carboxylate sesquihydrate) (available from Eisai Co., Ltd., Tokyo, Japan). E4021 is also known as 1-[4[(1,3-benzodioxol-5-ylmethyl)amino]-6-chloro-2-quinazolinyl]-4-piperidinecar-boxylic acid monohydrochloride CAS No: 150452-21-4 and has the formula: C₂₂H₂₂C₁₂N₄O₄ and the molecular weight: 477.34. E4021 is also known as 2-(4-Carboxypiperidino)-4-(3,4-methylenedioxybenzyl)amino-6-chloroquinazoline hydrochloride. This type V phospho-diesterase inhibitor can be made as described in U.S. Pat. No. 7,235,625 assigned to Palatin Technologies, Inc. The entire disclosure of this patent is herein incorporated by reference.

The type V phosphodiesterase inhibitor can be in the composition of the present application in an amount from about 0.05% w/w or w/v to about 40% w/w or w/v based on a total weight of the composition. In some embodiments, the type V phosphodiesterase inhibitor can be in the composition in an effective to amount to provide the mammal a dose of about 5 μg/kg to about 500 μg/kg.

For example, in one embodiment, the composition comprises E-4021, which is sodium 1-[6-chloro-4-(3,4-methylenedioxybenzyl)-aminoquinazolin-2-yl]piperidine-4-carboxylate sesquihydrate, that can be administered by injection at a dose of 50 mg, 100 mg, 150 mg, or 200 mg that can be administered 7 days or less (e.g., from about 30 minutes, about 45 minutes, about 90 minutes, about 1 day to about 7 days) prior to strenuous exercise.

The type V phosphodiesterase inhibitor can be administered as monotherapy in single or multiple doses or part of a dosage regimen with other agents. For example, the type V phosphodiesterase inhibitor can be administered as part of a treatment regimen with or without furosemide, aminocaproic acid, nitric oxide gas, aclidinium, albuterol, arformoterol, beclomethasone, budesonide, ciclesonide, clenbuterol, corticosteroids, dexamethasone, fluticasone, formoterol, indacaterol, bronchodilators (e.g., ipratropium bromide), levalbuterol, L-arginine, metaproterenol, mometasone, pirbuterol, salmeterol, tiotropium, nitroglycerin, isosorbide dinitrate, erythrityl tetranitrate, amyl nitrate, sodium nitroprusside, molsidomine, linsidomine chlorhydrate, vilanterol, non-steroidal anti-inflammatory drugs (NSAIDs), conjugated estrogens (e.g. Premarin®), antifibrinolytics (e.g. tranexamic acid), snake venom, aspirin, vitamin K, bioflavonoids (e.g., hesperidin-citrus bioflavioids), herbal remedies, concentrated equine serum omega-3 fatty acids, adrenergic blocking drugs (e.g., acepromazine), or a combination thereof before, during or after the type V phosphodiesterase inhibitor is administered to the mammal.

The type V phosphodiesterase inhibitor can be provided in a micronized powder form that optionally is lyophilized before it is mixed with a suitable solvent. In various embodiments, the particle size of the type V phosphodiesterase inhibitor can range from about 1 micron to 1000 microns. In some embodiments, the type V phosphodiesterase inhibitor can have a particle size of from about 5 microns to about 100 microns or from about 20 to 50 microns. In some embodiments, type V phosphodiesterase inhibitor can be mixed with one or more pharmaceutically acceptable solvents to form a liquid. A pharmaceutically acceptable solvent is non-toxic to recipients at the concentrations employed and is compatible with other ingredients of the composition. Suitable solvents to mix with the type V phosphodiesterase inhibitor include, but are not limited to, alcohol, water, saline, Ringer's solution, dextrose solution or the like.

### Organic Bases

In the current application, the type V phosphodiesterase inhibitor can be stabilized with an organic base. Suitable organic bases used in the current application are pharmaceutically acceptable and non-toxic to recipients at the concentrations employed and are compatible with other ingredients of the composition. Suitable organic bases or amino sugars include, but are not limited to, N-Acetylglucosamine, galactosamine, glucosamine, sialic acid, L-daunosamine, pyridine, alkanamines, such as methylamine, imidazole, benzimidazole, histidine, guanidine, phosphazene bases, hydroxides of quaternary ammonium cations, meglumine, L-arginine, triethylamine, diethylamine, ethanolamine, diethanolamine, triethanolamine, ethylenediamine, or a combination thereof.

In some embodiments, the organic base can be a basic amino acid or an amino sugar. In one embodiment, the organic base comprises meglumine, which can stabilize the type V phosphodiesterase inhibitor (e.g., E-4021). Meglumine is an amino sugar derived from glucose. Meglumine includes a compound with chemical formula H₃NHCH₂(CHOH)₄CH₂OH or C₇H₁₇NO₅, CAS Number 6284-40-8 and molecular weight of 195.21. Meglumine is also known as 1-Deoxy-1-methylaminosorbitol or N-Methyl-d-glucamine or 1-Deoxy-1-methylamino-D-glucitol. Meglumine includes derivatives and salts of meglumine. The derivatives and salts of meglumine include, but are not limited to, meglumine amidodrizoate, meglumine sodium amidodrizoate, meglumine cadopentetate, meglumine gadoterate, meglumine iotalamate, meglumine iotroxate, meglumine gadobenate, meglumine iodoxamate, meglumine flunixin, and gastrografin (meglumine sulfate). Products resulting from chemical modification of hydroxyl group, amino group, or others of the above-listed meglumines are also included in the meglumine of the present application.

In one embodiment, the organic base (e.g., meglumine) can be in the composition in an amount from about 0.05% w/w or w/v to about 40% w/w or w/v based on a total weight of the composition. In some embodiments, the organic base (e.g., meglumine) is in the composition in an amount of about 0.1% w/w or w/v to about 0.25%, about 0.3% to about 0.5%, about 0.75% to about 3%, or about 5% to about 20% w/w or w/v.

In one embodiment, the organic base (e.g., meglumine) can be in the composition in an amount of about 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, 4.5 to about 5% w/w or w/v based on the total w/w or w/v of the composition

The organic base is in the composition to aid the solubility of the type V phosphodiesterase inhibitor (e.g., E-4021) in the composition. The type V phosphodiesterase inhibitor can be soluble in a basic environment so that the organic base can raise the pH to the alkaline environment of about 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8. 9.9, 10, 10.1, 10.2, 10.3, 10.4, 10.5, 110.6, 10.7, 10.8, 10.9, 11.0, 11.1, 11.2, 11.3, 11.4, 11.5, 11.6, 11.7, 11.8, 11.9 to about 12.0 to solubilize and stabilize the type V phosphodiesterase inhibitor (e.g., E-4021).

The organic base (e.g., meglumine) can, among other things, stabilize the composition. Stabilize or stability with respect to storage is understood to mean that the type V phospho-diesterase inhibitor (e.g., E-4021) contained in the composition does not lose more than 20%, or more than 15%, or more than 10%, or more than 5% of its activity relative to activity of the composition at the beginning of storage. For example, when the organic base or amino sugar (e.g., meglumine) is added to the composition, the composition is stable at about 4° C. for at least about 18 months, where substantially no particulates or aggregates of the type V phosphodiesterase inhibitor are seen in the solution. In some embodiments, the organic base or amino sugar (e.g., meglumine) is added to the composition, the composition is stable at about 4° C. for at least about 24 months, where substantially no particulates or aggregates of the type V phosphodiesterase inhibitor are seen in the solution.

In some embodiments, the organic base or amino sugar (e.g., meglumine) is added to the composition, the composition is stable at about 6 months at room temperature, where substantially no particulates or aggregates of the type V phosphodiesterase inhibitor are seen in the solution. In some embodiments, the organic base or amino sugar (e.g., meglumine) is added to the composition, the composition is stable at about 6 months at 40° C. temperature, where substantially no particulates or aggregates of the type V phosphodiesterase inhibitor are seen in the solution.

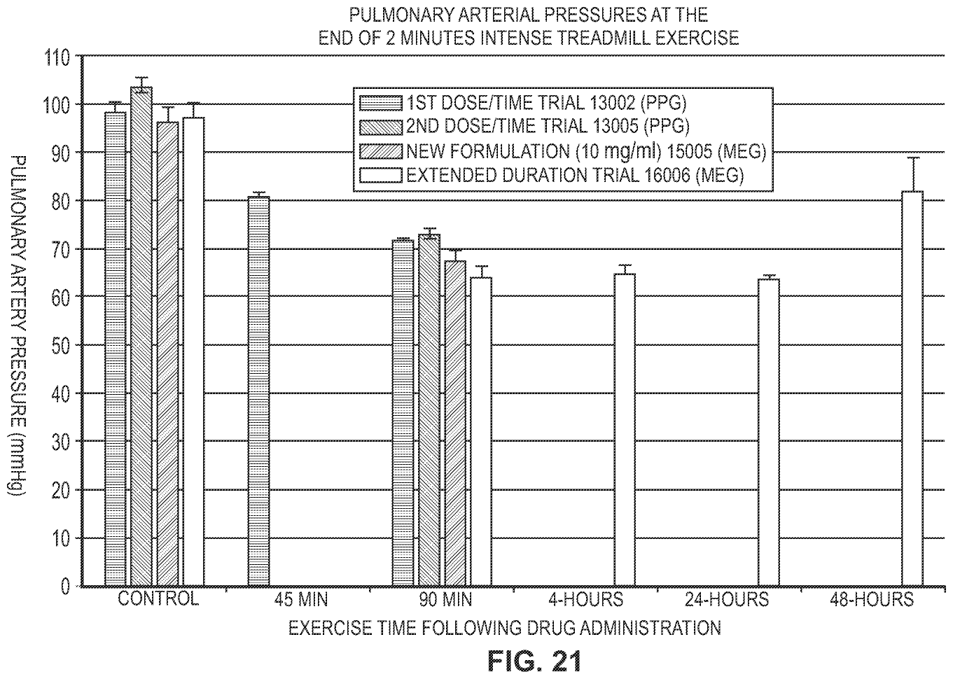

In some embodiments, the organic base (e.g., meglumine) can, among other things, extend the duration of action of the type V phosphodiesterase inhibitor (e.g., E-4021). Duration of action, in some embodiments, relates to the time throughout which the elevated pulmonary vascular pressure or EIPH is prevented, treated or reduced in the mammal. For example, pulmonary artery pressure (in FIG. 21) after the administration of type V phosphodiesterase inhibitor (e.g., about 100 mg of E-4021) containing meglumine shows that the duration of pulmonary artery pressure after exercise was reduced and the duration of this effect lasted at least 24 hours and was even lower than the control at 48 hours. There was also no need to go above the about 100 mg of E-4021 to observe this effect. However, the composition of the current application includes doses lower and higher than 100 mg depending on the mammal being treated, response by the mammal and parameters such as for example, age and weight of the mammal. Further, in some embodiments, the E-4021 did not increase or decrease markers of aerobic capacity or alter key marker of anaerobic metabolism, which would not give the mammal an advantage in racing.

While not wishing to be bound by one theory, it is believed that the organic base (e.g., meglumine) allows the type V phosphodiesterase inhibitor (e.g., E-4021) to have increased uptake into the cells and provides overall stability to the composition. In some embodiments, the organic base enhances stability and duration of action by at least 10%, at least 20%, at least 30%, at least 40%, at least 50%, at least 60%, at least 70%, at least 80%, at least 90%, or at least 100% compared to compositions that do not have the organic base.

In some embodiments, the type V phosphodiesterase inhibitor containing the organic base reduces pulmonary arterial pressure to about 90 mm Hg or less, about 30 minutes, 45 minutes, 90 minutes, 4 hours, 24 hours, 48 hours, 72 hours to about 96 hours after the type V phosphodiesterase inhibitor is administered to the mammal during an exercise event that produces pulmonary vascular pressure greater than 90 mm Hg.

In some embodiments, the organic base (e.g., meglumine) can, among other things, have reduced toxicity. For example, mammals receiving the type V phosphodiesterase inhibitor (e.g., E-4021) containing meglumine, did not have renal toxicity or blood in the urine as compared to mammals receiving the type V phosphodiesterase inhibitor (e.g., E-4021) containing propylene glycol. Therefore, in some embodiments, the compositions of the present application have a better safety profile.

The organic base (e.g., meglumine) used in the composition can be provided in a micronized powder form that optionally is lyophilized before it is mixed with a suitable solvent. In various embodiments, the particle size of the organic base (e.g., meglumine) can range from about 1 micron to 1000 microns. In some embodiments, the organic base (e.g., meglumine) can have a particle size of from about 5 microns to about 100 microns or from about 20 to 50 microns. Suitable solvents to mix with the organic base include, are pharmaceutically acceptable and non-toxic to recipients at the concentrations employed and are compatible with other ingredients of the composition. Suitable solvents include, but are not limited to, alcohol, water, saline, Ringer's solution, dextrose solution or the like.

In some embodiments, the compositions of the current application comprise sodium 1-[6-chloro-4-(3,4-methylenedioxybenzyl)-aminoquinazolin-2-yl]piperidine-4-carboxylate sesquihydrate), meglumine, alcohol, and water.

In some embodiments, the compositions of the current application consist essentially of sodium 1-[6-chloro-4-(3,4-methylenedioxybenzyl)-aminoquinazolin-2-yl]piperidine-4-carboxylate sesquihydrate), meglumine, alcohol, and water.

In some embodiments, the compositions of the current application consists of sodium 1-[6-chloro-4-(3,4-methylenedioxybenzyl)-aminoquinazolin-2-yl]piperidine-4-carboxylate sesquihydrate), meglumine, alcohol, and water.

One exemplary embodiment of the composition is an injectable composition that comprises E-4021, which is sodium 1-[6-chloro-4-(3,4-methylenedioxybenzyl)-aminoquinazolin-2-yl]piperidine-4-carboxylate sesquihydrate at a dose of 50 mg, 100 mg, 150 mg, or 200 mg, meglumine in an amount of 25 mg, dehydrated alcohol in an amount of 3.94 g, and water for injection.

In some embodiments, the compositions of the present application may be provided in one or more vials, ampules, prefilled syringes, bottles, bags, and/or other containers. In some embodiments, the compositions, vials, ampules, prefilled syringes, bottles, bags, and/or other containers can be sterilized and/or preservative free.

The compositions of the present application may contain acceptable carriers, excipients, that are nontoxic to recipients and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenyl, butyl or benzyl alcohol; alkyl parabens such as methyl or propyl paraben; catechol; resorcinol; cyclohexanol; 3-pentanol; and m-cresol); monosaccharides, disaccharides, and other carbohydrates including glucose, mannose, dextrose or dextrins; chelating agents such as EDTA; sugars such as sucrose, mannitol, trehalose or sorbitol; salt-forming counter-ions such as sodium; metal complexes (e.g., Zn-protein complexes) and/or water.

The present application, in some embodiments, also provides a kit for preventing or treating exercise-induced pulmonary hemorrhage or elevated pulmonary vascular pressure in a mammal in need thereof, the kit comprising an aqueous composition comprising a type V phosphodiesterase inhibitor as discussed above, meglumine as discussed above, alcohol and water.

In some embodiments, the kit further includes diluent and an administration vehicle to administer the composition to the mammal, the diluent or administration vehicle can be, for example, sodium chloride, dextrose, phosphate buffered saline, sterile water for injection or a combination thereof. The kit can also have instructions for use and have packaging enclosing the components of the kit in a sterile condition. The kit can further include a syringe, needle, disinfectant swabs, and/or a vial sterilized to help administer the composition.

### Methods of Making the Composition

In some embodiments, a method of making a composition is provided, the method comprising adding the organic base discussed above to the type V phosphodiesterase inhibitor discussed above to form the composition. The order of addition and mixing is not critical, therefore, in some embodiments, a method of making a composition is provided, where the type V phosphodiesterase inhibitor discussed above is added to the organic base discussed above to form the composition.

In one embodiment, the organic base (e.g., meglumine) used in the composition can optionally be micronized and optionally lyophilized before it is mixed with a suitable solvent. Suitable solvents include, but are not limited to, alcohol, water, saline, Ringer's solution, dextrose solution or the like. The organic base, such as meglumine, is mixed with a suitable solvent such as water. The organic base will form an alkaline solution or suspension (e.g., pH about 10.4), which will be ideal for mixing the type V phosphodiesterase inhibitor discussed above (e.g., E-4021). To the alkaline solution or suspension, the type V phosphodiesterase inhibitor (e.g., E-4021) can then be added and another suitable solvent, such as alcohol can be added to that solution or suspension to form the composition. Water can then be added to the final composition to form the injectable solution.

In one embodiment, the alcohol or other solvent can be in the composition in an amount of about 1% w/w or w/v, 2, 3, 4, 5, 10, 15, 20, 25, 30, 35, 40, 45, 50, 55, 60, to about 65% w/w or w/v based on a total weight of the composition.

The mixing and additions of solvents and powders can be done under aseptic conditions and the final solution can be filtered to form a sterilized injectable product. Typical pH of the final solution for injection can be alkaline or about 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8. 9.9, 10, 10.1, 10.2, 10.3, 10.4, 10.5, 110.6, 10.7, 10.8, 10.9, 11.0, 11.1, 11.2, 11.3, 11.4, 11.5, 11.6, 11.7, 11.8, 11.9 to about 12.0 to solubilize and stabilize the type V phosphodiesterase inhibitor (e.g., E-4021).

In some embodiments, one or more components of the composition and/or the device (e.g., vial, syringe, etc.) to administer the composition may be sterilizable by radiation in a terminal sterilization step in the final packaging. In various embodiments, gamma radiation can be used in the terminal sterilization step, which involves utilizing ionizing energy from gamma rays that penetrates deeply in the packaging. Gamma rays are highly effective in killing microorganisms, they leave no residues nor have sufficient energy to impart radioactivity to the packaging. Gamma rays can be employed when the composition and/or the device is in the package and gamma sterilization does not require high pressures or vacuum conditions, thus, package seals and other components are not stressed.

In some embodiments, the composition and/or the device (e.g., vial, syringe, etc.) may be packaged in a moisture resistant package and then terminally sterilized by gamma irradiation. In use, the practitioner removes the one or all components from the sterile package.

In some embodiments, the composition and/or the device (e.g., vial, syringe, etc.) may be sterilized using electron beam (e-beam) radiation. E-beam radiation comprises a form of ionizing energy, which is generally characterized by low penetration and high-dose rates. E-beam irradiation is similar to gamma processing in that it alters various chemical and molecular bonds on contact, including the reproductive cells of microorganisms. Beams produced for e-beam sterilization are concentrated, highly charged streams of electrons generated by the acceleration and conversion of electricity.

Other methods may also be used to sterilize the composition and/or the device (e.g., vial, syringe, etc.), including, but not limited to, gas sterilization, such as, for example, with ethylene oxide or steam sterilization.

### Methods of Administering the Composition

In some embodiments, the type V phosphodiesterase inhibitor (e.g., E-4021) containing the organic base (e.g., meglumine) can be used to prevent or treat exercise-induced pulmonary hemorrhage or elevated pulmonary vascular pressure in a mammal. For example, 7 days, 5 days, 4 days, 3 days, 2 days, 1 day, 8 hours, 4 hours, 90 minutes, 45 minutes, 30 minutes, 15 minutes, 10 minutes, 5 minutes, or 1 minute before exercise, the type V phosphodiesterase inhibitor can be administered to the mammal. The mammal's pulmonary arterial pressure will be reduced to about 90 mm Hg or less, about 30 minutes, 45 minutes, 90 minutes, 4 hours, 24 hours, 48 hours, 72 hours to about 96 hours after the type V phosphodiesterase inhibitor is administered to the mammal during an exercise event that produces pulmonary vascular pressure greater than 90 mm Hg.

Multiple techniques of administering the compositions of the present application exist in the art including, but not limited to, administration by intravenous infusion, intravenous push, intramuscular, subcutaneous, intraperitoneal, intraarterial, inhalation, intradermal, oral, topical, or ophthalmic administration.

The compositions of the present application can be administered as a single dose injection. The injectable compositions can also be administered in multiple injection doses such as, for example, 1, 2, 3, 4, 5 or more injections per day, per week, per month or every six months depending on the severity of the condition, response to treatment or extent of prophylaxis. For example, as the mammal's lung tissue heals, the frequency of administration and/or time interval can decrease as well. The injectable composition can be administered via IV push over a period of less than 5 minutes. The compositions of the current application can be administered by an intravenous infusion to the mammal, for example, using an infusion pump.

In some embodiments, the compositions of the present application can be administered as one dose to a racehorse prior to the race.

In some embodiments, the compositions of the present application can be mixed with suitable diluent and/or vehicle for delivery to the mammal. These include, but are not limited to, sodium chloride, dextrose, phosphate buffered saline, sterile water for injection or a combination thereof.

Mammals being treated according to the present application may also be treated with one or more additional therapeutic agents. In certain embodiments, the type V phosphodiesterase inhibitor can be administered as part of a treatment regimen with furosemide, aminocaproic acid, nitric oxide gas, aclidinium, albuterol, arformoterol, beclomethasone, budesonide, ciclesonide, clenbuterol, corticosteroids, dexamethasone, fluticasone, formoterol, indacaterol, bronchodilators (e.g., ipratropium bromide), levalbuterol, L-arginine, metaproterenol, mometasone, pirbuterol, salmeterol, tiotropium, nitroglycerin, isosorbide dinitrate, erythrityl tetranitrate, amyl nitrate, sodium nitroprusside, molsidomine, linsidomine chlorhydrate, vilanterol, non-steroidal anti-inflammatory drugs (NSAIDs), conjugated estrogens (e.g. Premarin®), antifibrinolytics (e.g. tranexamic acid), snake venom, aspirin, vitamin K, bioflavonoids (e.g., hesperidin-citrus bioflavioids), herbal remedies, concentrated equine serum omega-3 fatty acids, adrenergic blocking drugs (e.g., acepromazine), or a combination thereof before, during or after the type V phosphodiesterase inhibitor is administered to the mammal.

Having now generally described the invention, the same may be more readily understood through the following reference to the following examples, which are provided by way of illustration and are not intended to limit the present invention unless specified.

### EXAMPLES

### Example 1: E-4021 with Propylene Glycol Injection

This study was performed to develop a formulation as a 50 mL single dose vial with the following ingredients:

TABLE 1
Propylene Glycol Formula
50 mL Single Dose Vial (13002 and 13005)
Preservative Free

| | | |
|---|---|---|
| Ingredient: | Per 50 mL | Weight % |

| | | | |
|---|---|---|---|
| E-4021 | 100 | mg | 0.2 |
| Propylene Glycol, USP | 23. | g | 46 |
| Dehydrated Alcohol, USP | 17.7 | g | 35.4 |

| | | |
|---|---|---|
| Water for Injection, USP | Q.s to 50 mL | 10 |

The drug product vehicle contains about 35-45 wt % alcohol, 46 wt % propylene glycol and 10 wt % WFI. Target Animal Safety (TAS) Study was suspended due to toxicity at higher (e.g., 4×) doses. Test doses (in horses) with the vehicle confirmed that the dose toxicity was due to the vehicle. More specifically, propylene glycol was believed to be the cause of toxicity. The toxicity included blood in the urine which could indicate kidney damage.

### Example 2: Development of a Formula without Propylene Glycol to Reduce Toxicity

This study was performed to develop a formulation without propylene glycol as a 10 mL single dose vial with the following ingredients:

TABLE 2
Dehydrated Alcohol Formula
E-4021 Injection
10 mL Single Dose Vial
Preservative Free

| | | |
|---|---|---|
| Ingredient: | Per 10 mL | Weight % |

| | | | |
|---|---|---|---|
| E-4021 | 100 | mg | 1 |
| Dehydrated Alcohol, USP | 3.94 | g | 39.4 |

| | |
|---|---|
| Water for Injection, USP | Q.s to 10 mL |

The formulated product (100 mg/10 mL) was found to be soluble in 50% ETOH (pH=11). Clinical samples of E-4021 for injection, 10 mg/mL (pH 11) in 50% ethanol, were prepared for GLP dose confirmation study. The remainder of the lab batch was placed on accelerated stability. NaOH was used to adjust the pH. Stability studies found that the product developed particulate matter at three months storage at 4° C., 25° C. and 40° C. This indicates that the product had reduced stability.

### Example 3: Development of a Formula with Meglumine, an Organic Base Commonly Used as a Buffer/Stabilizing Agent in FDA Approved Equine Products

This study was performed to develop a formulation with meglumine as a 10 mL single dose vial with the following ingredients to reduce toxicity and to improve stability:

TABLE 3
Meglumine Formula
10 mL Single Dose Vial (15005 and 16006)
Preservative Free

| | | |
|---|---|---|
| Ingredient: | Per 10 mL | Weight % |

| | | | |
|---|---|---|---|
| E-4021 | 100 | mg | 1 |
| Meglumine, NF | 25 | mg | 0.25 |
| Dehydrated Alcohol, USP | 3.94 | g | 3.94 |

| | |
|---|---|
| Water for Injection, USP | Q.s to 10 mL |

Meglumine was used to adjust the pH of the formulation instead of NaOH which was used in Example 2. The drug product has been found to be stable at 4° C. Retained samples stored at 4° C. for 18 months remain particle free. On the other hand, samples stored for 3 months at RT and 40° C., develop near visible particles that have been identified as aggregates of E-4021. This indicates that the meglumine, an organic base, improves the stability of the formulation. It is also non-toxic to horses.

### Example 4: Formulation with Meglumine

In this example, each mL of the composition for injection comprises, consists essentially of or consists of 10 mg of E-4021, 2.5 mg/mL meglumine and 0.5 mL ethyl alcohol in quantity sufficient water for injection. 10 mL can be stored in 10 mL vials to provide an injectable composition which has been tested as indicated in Table 6 below.

TABLE 6

| TEST | TEST SPECIFICATIONS | RESULTS |
|---|---|---|
| Description | Clear, colorless to pale | Complies |
| | yellow clear solution with | |
| | no visible particles | |
| pH <791> | 10.0-11.2 | 10.8 |
| Assay | 90.0-110.0% | 99.7% |
| Particulate Matter | ≤6000 Particles/vial | 71 per container |
| (USP 40 <788>) | for ≥10 μm | |
| | ≤600 Particles/vial | 45 per container |
| | for ≥10 μm | |
| Chromatographic | Not More than ≤0.3% | <0.3% |
| Purity - Total | | |
| Sterility | Complies with the | Pass |
| USP 40<71> | test for sterility | |
| Bacterial Endotoxin | <10.0 EU/mL | <1.00 EU/mL |
| (USP 40 <788>) | | |

The above composition can be stored under refrigeration at 2-8° C. (36-46° F.). Samples removed for clinical testing may be stored for up to two weeks at 20-25° C. (68-77° F.) with excursions permitted to I5-30° C. (59-86° F.). The composition has the following properties illustrated in Table 7 below.

TABLE 7

| TEST | TEST SPECIFICATIONS | RESULTS |
|---|---|---|
| Sterility | Complies with the | Pass |
| (USP 40<71>) | test for sterility | |
| Sterility Suitability | Conforms | Pass |
| (USP 40<71>) | | |
| Particulate Matter | ≤6000 Particles/vial | 71 per container |
| (USP 40 <788>) | for ≥10 μm | |
| | ≤600 Particles/vial | 45 per container |
| | for ≥25 μm | |
| Bacterial Endotoxin | <10.0 EU/mL | <1.00 EU/mL |
| (USP 40 <85>) | | |
| Inhibition/Enhancement | No Interference | Pass |
| (USP 40 <85>) | | |

### Example 5: Dose Selection of a Novel Type 5 Phosphodiesterase Inhibitor for a Strenuously Exercising Equine Using a Treadmill

The purpose of this study was to determine the effects of intravenous administration of E-4021 on cardiorespiratory variables in exercising horses to facilitate the selection of a product dose for the control of hemorrhage associated with Exercise Induced Pulmonary Hemorrhage (EIPH). The formulation given was the propylene glycol formulation of Table 1.

Animals: The Rutgers University Institutional Animal Care and Facilities Committee approved all methods and procedures used in this experiment. Eight mature unfit Standardbred horses, 4 geldings and 4 mares were used in this study. The horses were healthy and acceptable for the study as determined by the study veterinarian. All horses underwent a physical examination to establish suitability for inclusion within the study. This included a physical examination and body weights. All of the horses were dewormed and vaccinated per standard veterinary practice. Animals were fed a maintenance ration of alfalfa/grass hay ad libitum and concentrate as needed. Water and mineral blocks were provided ad libitum. The horses were housed individually in 3×3 m stalls between 1600 to 0700 hours. They then performed their daily exercise followed by turnout in groups of 4 in 2 acres dry lot paddocks for ˜7 hrs/day.

General Study Design: There were four phases in this study detailed below. The horses performed conditioning exercise 4 d/week in a motorized equine exercise machine (Equi-cizer, Calgary, Canada) and heavy exercise 1 d/week on a high-speed treadmill (Sato I, Lexington Ky.) during Phase I. Heavy exercise prepared the horses for the later exercise tests performed during Phase Ib, Phase II, and Phase III of the experiment. The exercise tests were performed in the place of the heavy exercise training during those later three periods.

Phase Ia (Conditioning and Training; Weeks 1-8): Eight horses followed a standard exercise procedure (SEP) with the exception that the galloping speed for each horse was to be increased each week up to a safe maximal intensity for each horse. This was determined partially objectively (horses are capable of maintaining their running position on the treadmill with encouragement) and partially subjectively (horse handler's skilled observation of level of fatigue).

The purpose of this conditioning period was to get all eight horses to a fitness level at which they would have reproducible oxygen consumption, carbon dioxide production and heart rate at maximal (heavy) exercise intensity. The length of this period was based upon the documented fact that the vast majority of treadmill trained horses usually reach a consistent fitness level within an 8-week training period, although some take longer.

TABLE 4
Treatment groups A & B and their
Standard Exercise Procedure (SEP)

| | GROUP A | GROUP B |
|---|---|---|
| WEEKDAYS | 2 Geldings & 2 Mares | 2 Geldings & 2 Mares |
| SUNDAY | Paddock (free-exercise) | Paddock (free-exercise) |
| MONDAY | Light (walk, trot, canter) | Light (walk, trot, canter) |
| TUESDAY | Moderate (walk, trot, | Light (walk, trot, canter) |
| | canter, slow gallop) | |
| WEDNESDAY | Light (walk, trot, canter) | Moderate (walk, trot, |
| | | canter, slow gallop) |
| THURSDAY | Heavy (walk, trot, canter, | Light (walk, trot, canter) |
| | max gallop (as tolerated) | |
| FRIDAY | Light (walk, trot, canter) | Heavy (walk, trot, canter, |
| | | max gallop (as tolerated) |
| SATURDAY | Paddock (free-exercise) | Paddock (free-exercise) |

Phase Ib (Weeks 9-12): During this period, all eight horses performed three incremental exercise tests (GXT) to document that the horses had reached an acceptable and stable level of fitness. Stability was documented by demonstrating that the oxygen consumption did not differ by more than 10% over the course of the three GXTs that were performed 2 weeks apart. The GXTs used previously published methods to measure maximal oxygen uptake (VO₂ₘₐₓ) and indices of exercise performance (Rose et al., 1988; Seehernan and Morris, 1990; Birks et al., 1991; Kearns and McKeever, 2002; Streltsova et al., 2006; McKeever et al., 2006; Liburt et al., 2009). The horses were weighed just prior to the test. During the incremental exercise tests, the horses ran on a high-speed horse treadmill (Sato I, Equine Dynamics, Inc., Lexington, Ky.) at a fixed 6% grade. Horses wore an indirect open-flow calorimeter apparatus (Oxymax-XL, Columbus Instruments, Inc., Columbus, Ohio) to measure oxygen uptake and carbon dioxide production. The GXTs started at an initial speed of 4 m/s for 1 minute. Speed was then increased to 6 m/s, followed by incremental increases of 1 m/s every 60 s (omitting 5 m/s) until the horses reached fatigue. Fatigue was defined as the point where the horse could not keep up with the treadmill despite humane encouragement. At the point of fatigue, the treadmill was stopped, and 5 min of post-exercise data recorded. Oxygen uptake was measured continuously during the test and recorded at 10 s intervals using the open flow calorimetry system.

Phase II: This was the first part of the main study with test-article dosing of the horses (Weeks ≥12). This part of the experiment was conducted using a randomized semi-crossover design with each horse undergoing a control round within the first two weeks of the phase. Horses were randomly assigned to one of five treatments (CON; 50-45; 100-45; 50-90; 100-90). Control (CON) where no drug was administered; 50-45 where they were tested 45 minutes after receiving a dose of 50 mg; 100-45 where they were tested 45 minutes after receiving 100 mg of the test-article; 50-90 where they were tested 90 minutes after receiving a 50 mg dose; and 100-90 where they were tested 90 minutes after receiving a 100 mg dose. For this phase, the horses ran a simulated race test (SRT) every week on the heavy exercise days. Group A (n=4) horses ran their tests on Thursdays (mornings) and Group B (n=4) horses ran their tests on Fridays (mornings). All horses continued with light and moderate exercise as indicated for the other study days.

Phase III: This was the second part of the main study. This part of the experiment was conducted using a randomized crossover design where horses were initially assigned to one of four treatments (CON-B; 100-90B; 150-90; 200-90). Control (CON-B) where the horses received no drug; 100-90B where they were tested 90 minutes after receiving a 100 mg dose; 150-90 where they were tested 90 minutes after receiving a 150 mg dose; and 200-90 where they were tested 90 minutes after receiving a 200 mg dose. As with the other treatment phase the horses ran a weekly simulated race test (SRT) on the heavy exercise days. Group A (n=4) horses ran their tests on Thursdays (mornings) and Group B (n=4) horses ran their tests on Fridays (mornings).

Simulated Race Test (SRT): During the SRT, each horse ran at a speed calculated to correspond to 110% of the speed required to produce maximal oxygen uptake as measured during the GXTs performed during Phase Ib. The test was conducted on the treadmill at a fixed 6% grade, and consisted of a 2-minute warm-up at 4 m/s; a run for 2 minutes at the individualized speed calculated to correspond to 110% of the speed required to elicit VO₂ₘₐₓ; followed by a 2-minute recovery at 2 m/s. Hemodynamic measurements were recorded, and blood samples were obtained to correspond with the end of the warm-up, at 1 minute and 2 minutes of the high-speed run, and at the end of the recovery period.

Acute Animal Preparation: On the morning of each trial 4 horses were brought into the treadmill barn and placed in stalls where they were catheterized and instrumented. All SRTs were conducted between 0800 and 1200 hours. The mean room temperature of the lab during exercise was 21.1° C. Before the test, the horses were weighed and catheters were inserted percutaneously into the left (14 gauge, Angiocath, Becton Dickenson, Parsippany, N.J.) and right (8.0 F catheter introducer, Argon Medical, Plano, Tex.) jugular veins, respectively, using sterile techniques and local lidocaine anesthesia. The horses were then instrumented. A thermistor probe (IT-24P, Physiotemp, Clifton, N.J.) was inserted through the left jugular catheter for the measurement and recording (Model #Bat-10, Physiotemp, Clifton, N.J.) of core body temperature. A fluid filled PE180 tube was passed through the catheter introducer with its end position approximately 5 cm beyond the pulmonary valve to measure pulmonary arterial (PA) pressure. The position of the catheter was verified before and after exercise using the waveform recorded on the hemodynamic recording system (DTXPlus transducers, Argon Medical Devices, Plano, Tex.; with pressures recorded using a commercial A/D system, WinDaq, Dataq Instruments, Akron, Ohio).

Cardiovascular measurements made in each trial included pulmonary artery pressure, as discussed above, and continuous ECG recording (base-apex ECG signals recorded using a commercial system; Televet 100, Langeskov, Denmark) for evaluation of heart rate, rhythm and ECG morphology.

Blood samples were collected anaerobically into heparinized 3 mL syringes. Samples were used to measure blood gas variables (PpaO₂, PpaCO₂, pH, sO₂), as well as the concentrations of Na+, K+, CA++, lactate, glucose, hemoglobin, and packed cell volume. Blood gases and chemistries were measured using a Radiometer ABL 880 Flex analyzer. Packed cell volume was measured using the microhematocrit technique. Blood gases were temperature corrected using the core temperature recorded during the exercise test.

Oxygen consumption and carbon dioxide production were measured every 10 seconds using the open flow indirect calorimeter (Oxymax-XL, Columbus Instruments, Columbus, Ohio).

Statistical analysis: Data were subjected to ANOVA for repeated measures using a commercial software package (JMP, SAS Institute Inc., Cary, N.C.). A Dunnett test was used to detect differences from baseline values within treatments, and the Tukey HSD post-test was used to detect differences between groups at each data collection point. Values of P<0.05 were considered significant.

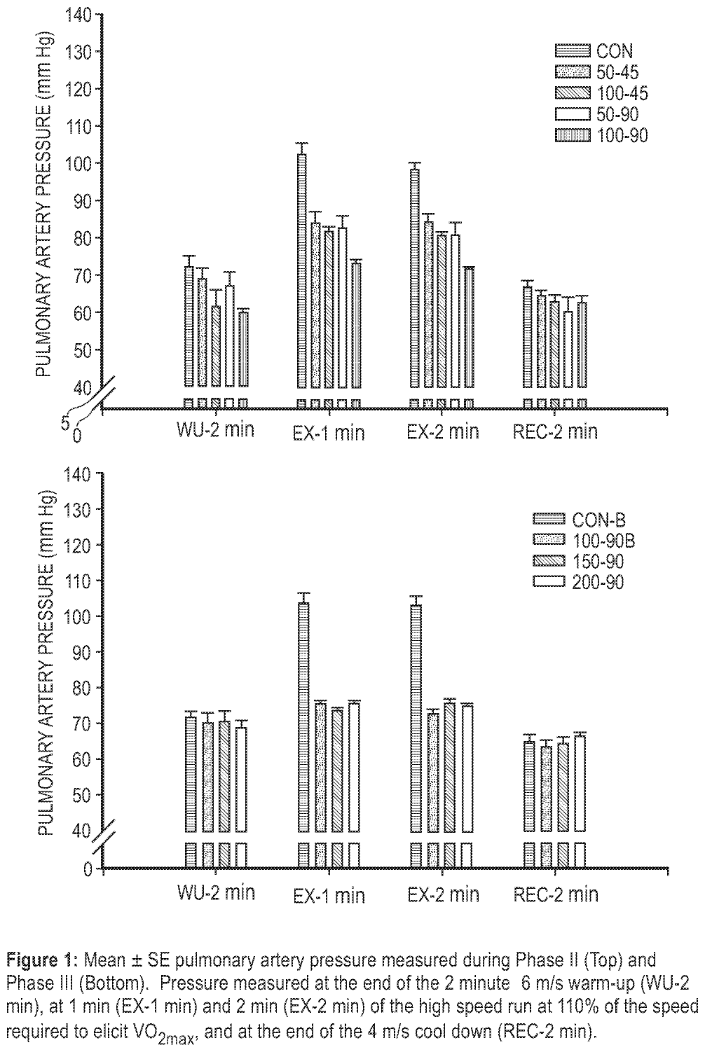

Results: Exercise resulted in a dramatic and significant increase in pulmonary artery pressure during all trials (FIG. 1). The magnitude of the increase seen in the present study is consistent with previously published data on exercising horses (Kearns and McKeever, 2002). Exercise also resulted in changes in blood gases and other blood parameters that were consistent with recognized responses to exercise in horses (Rose et al., 1988; Seehernan and Morris, 1990; Birks et al., 1991; Kearns and McKeever, 2002; Streltsova et al., 2006; McKeever et al., 2006; Liburt et al., 2009).

Pulmonary Artery Pressure: The major finding of the present study was that pulmonary artery pressures were substantially and significantly lower during intense exercise when the horses received E-4021. This was most apparent at the 2-minute point of the high intensity run in both Phase II and Phase III. In Phase II, the 100 mg dose given at 90 minutes prior to exercise resulted in the lowest PA pressure during exercise (P<0.05). Phase III was conducted to see if an increase in the dose given 90 minutes prior to exercise would result in an even lower exercise-related PA pressure. At 2 minutes of high intensity exercise, mean pulmonary artery pressures were lower (P<0.05) during the runs where the horses received E-4021 compared to control (FIG. 1). However, there was no difference (P>0.05) in the magnitude of PA pressure measured during the 100 mg vs. 150 mg vs. 200 mg trials (FIG. 1). Another important observation was the fact that the responses measured during the two control runs (CON and CON-B) were virtually identical (P>0.05) as were the responses measured during the two 100 mg runs (100-90 and 100-90B) (P>0.05). Finally, the ˜30 mm Hg lower PA pressure after 2 min intense exercise, seen with the 100 mg dose given at 90 min in both Phase II and III, represents a substantial and clinically significant lower exercise-related PA pressure.

Markers of Performance: The second major observation of the present study was that E-4021 did not alter key markers of aerobic and anaerobic performance. Those key markers include the rate of oxygen consumption, plasma lactate concentration.

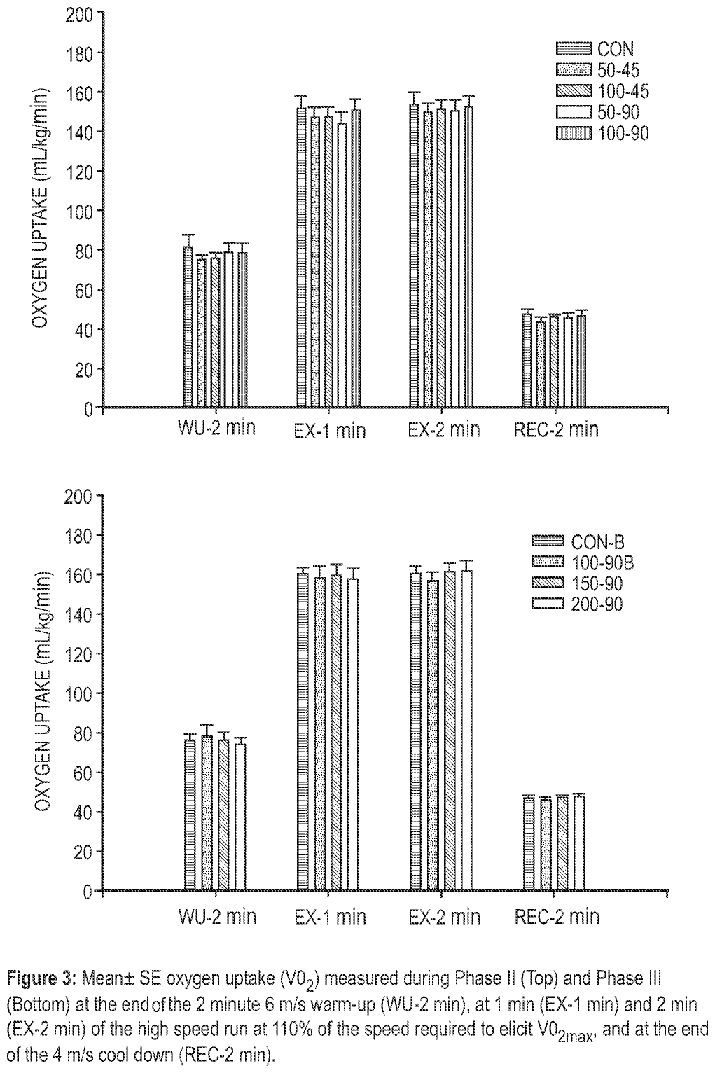

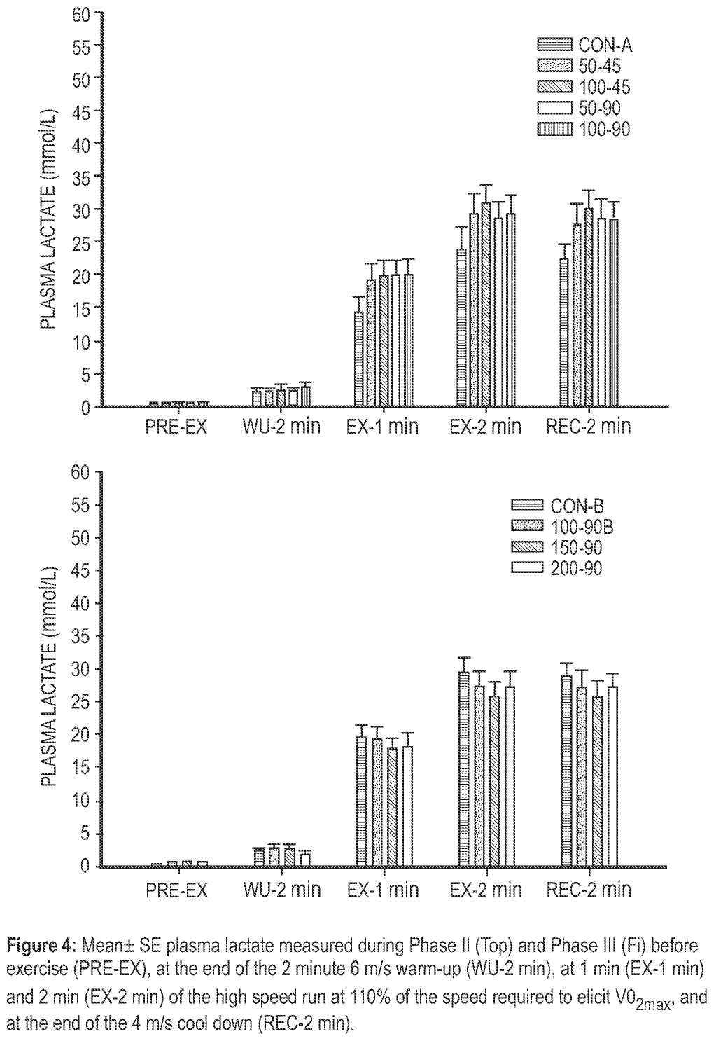

The maximal rate of oxygen consumption (VO₂ₘₐₓ) is a key marker of aerobic capacity. Using the Fick equation, we know that oxygen uptake can be expressed by the formula: VO₂=CO×(a−v) O₂. Cardiac output (CO) and the arterial content of oxygen give insight into central mechanism of oxygen delivery. In the horse, splenic contraction at the onset of exercise mobilizes up to 12 liters of red blood cell rich blood into the central circulation. This volume load contributes greatly to the increase in pulmonary artery pressure observed during exercise. The increase in volume enhances CO and the extra red blood cells increase the arterial O₂ content. Combined, this enhances the ability to transport oxygen. Separately, the arterial-venous oxygen content difference [(a−v) O₂] gives us insight into peripheral mechanisms affecting the extraction and utilization of oxygen. Anything affecting hemodynamics has the potential to increase or decrease this key marker of aerobic performance. The SRT protocol in this study used a velocity calculated to correspond to a speed 110% of the speed that was required to elicit VO₂ₘₐₓ documented in the incremental exercise tests (GXT) performed in Phase Ib. During the SRTs we observed a significant and expected increase in oxygen consumption reflecting the demand of exercise. Furthermore, the horses had a mean VO₂ observed at 1 and 2 minutes of the high intensity portion of the SRT that were identical to the mean values for VO₂ₘₐₓ measured during the GXTs performed in Phase Ib. Importantly, there was no effect of E-4021 on VO₂ (FIG. 3) measured during the SRTs performed in Phase II and Phase III. Put another way, E-4021 did not increase or decrease this marker of aerobic capacity. Similarly, there was a substantial effect of high intensity exercise (P<0.05) on plasma lactate concentration during the SRTs. However, there was no effect (P>0.05) of E-4021 on plasma lactate concentration suggesting the drug did not alter a key marker of anaerobic metabolism (FIG. 4).

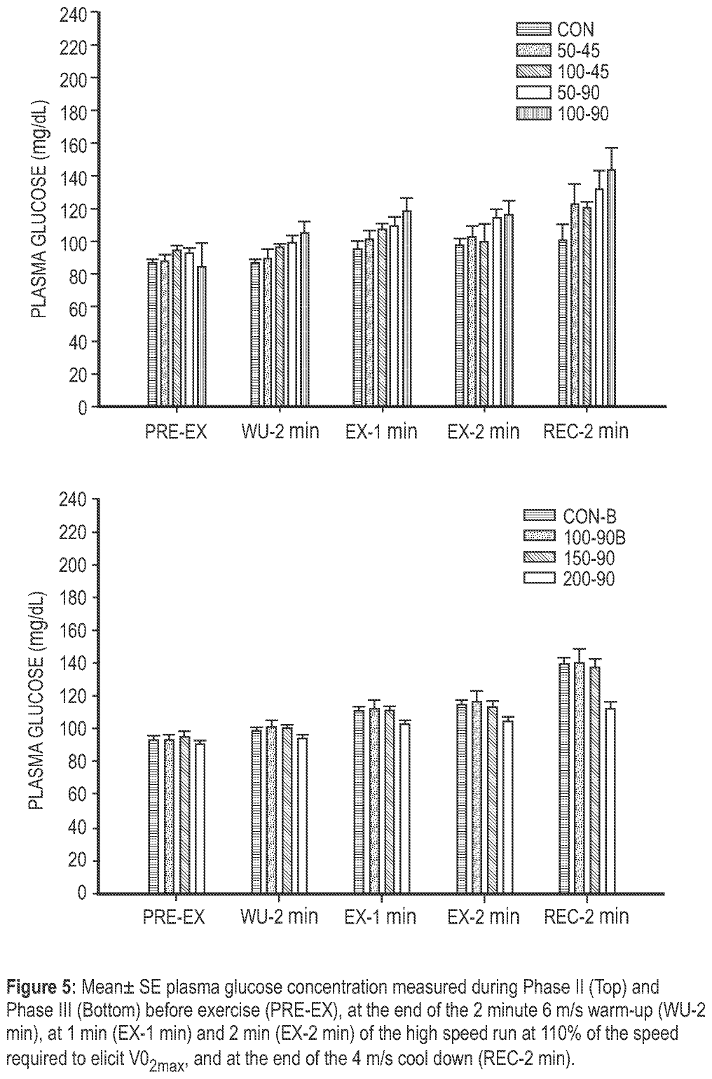

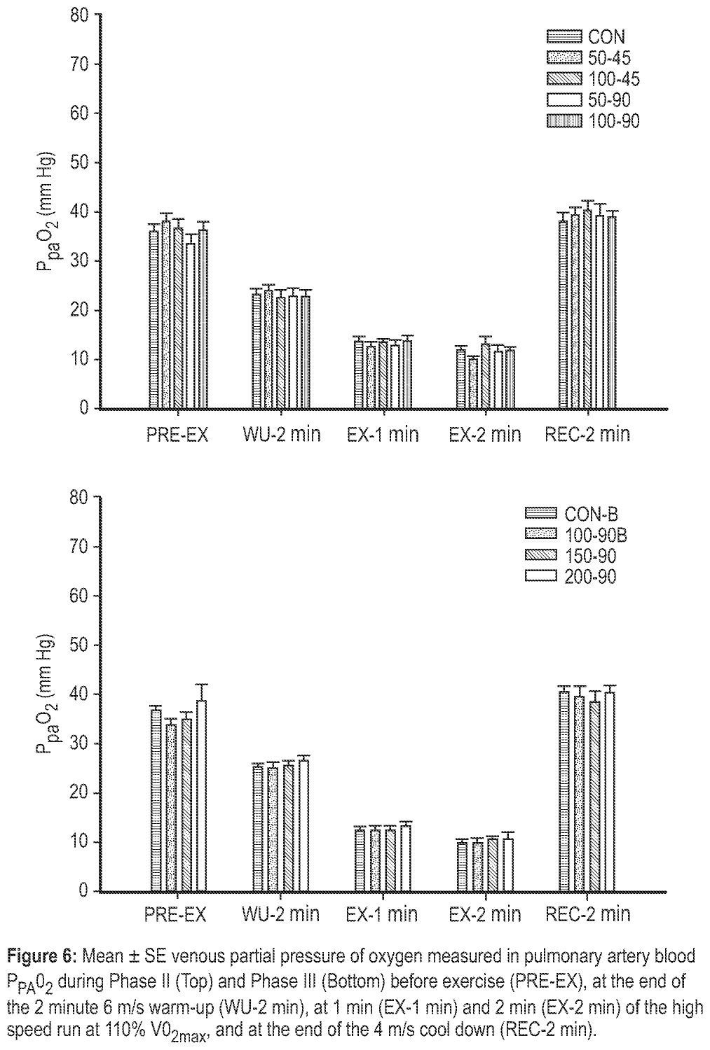

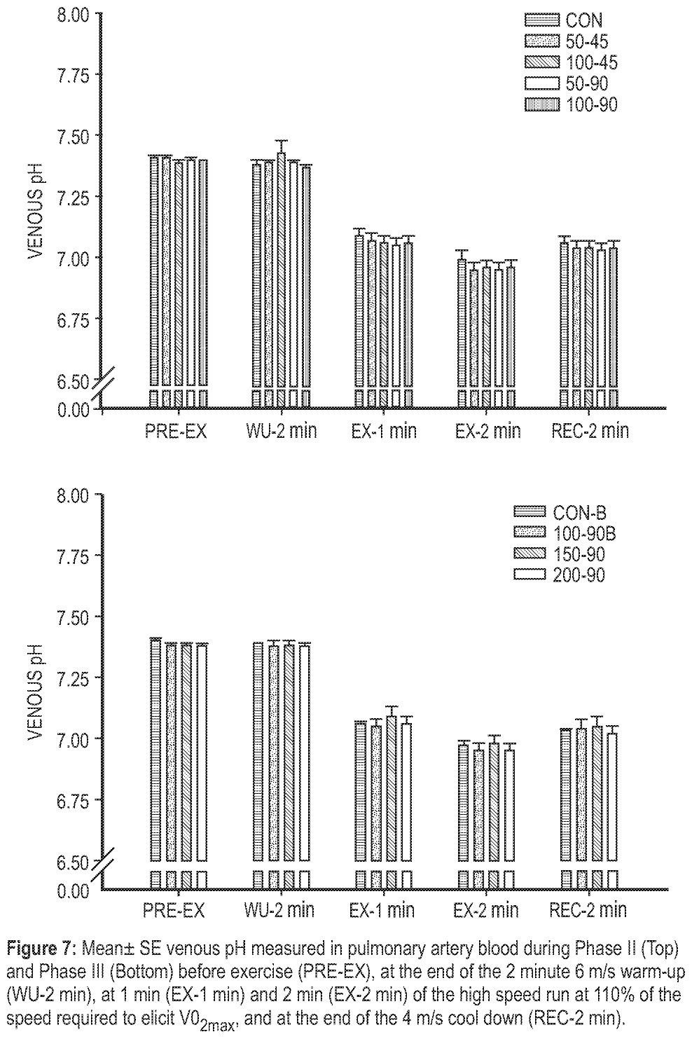

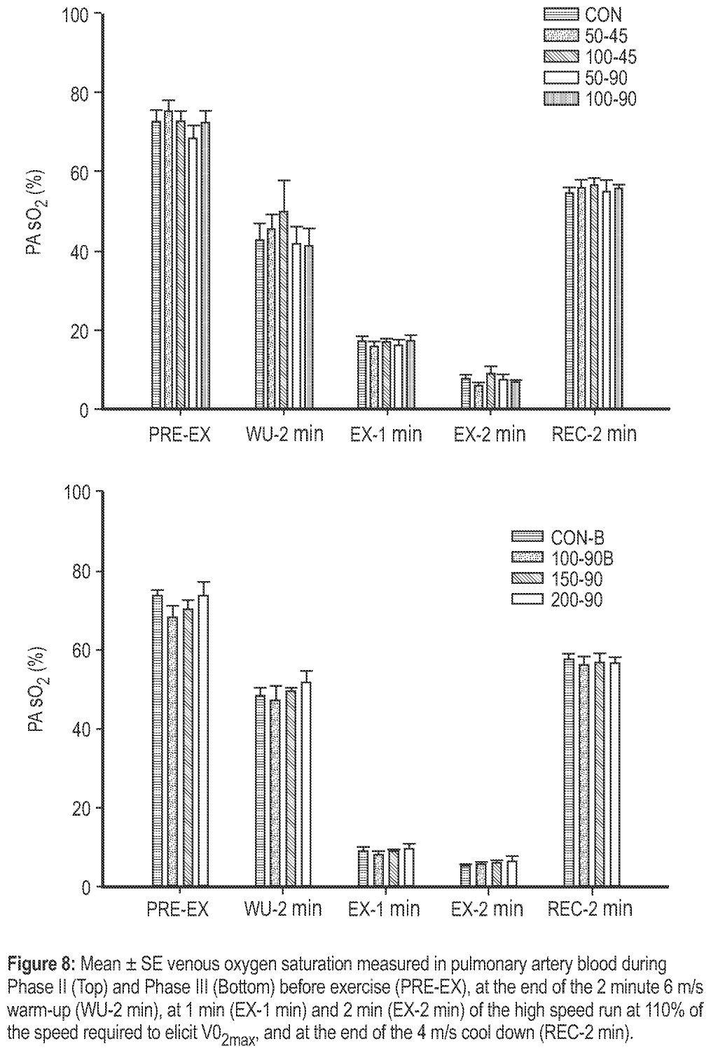

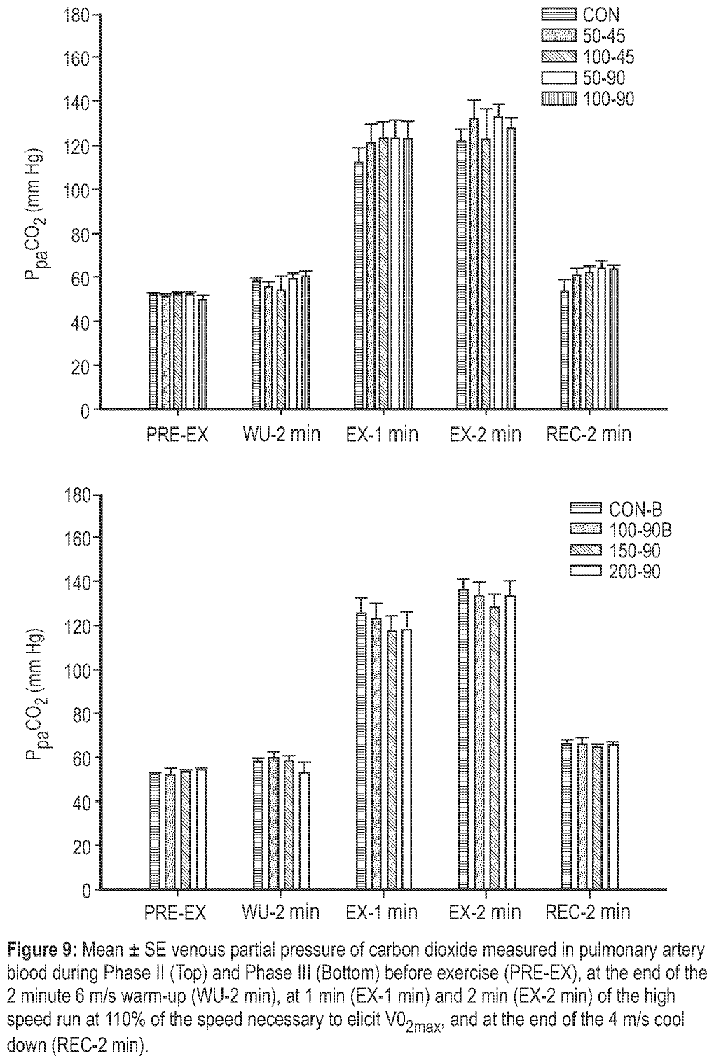

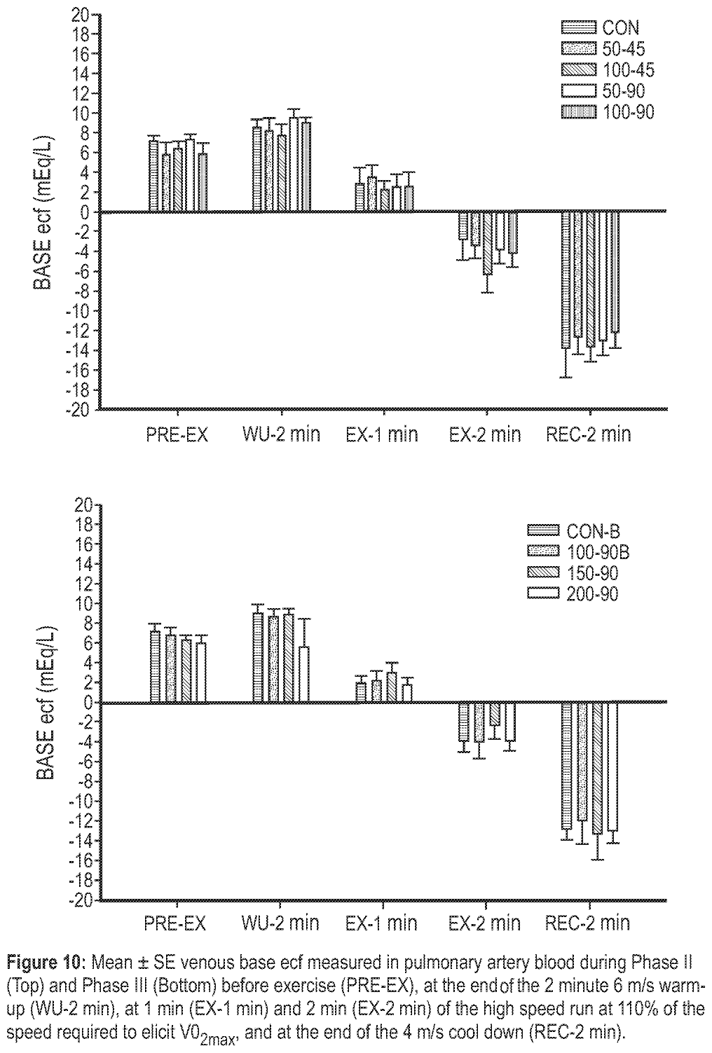

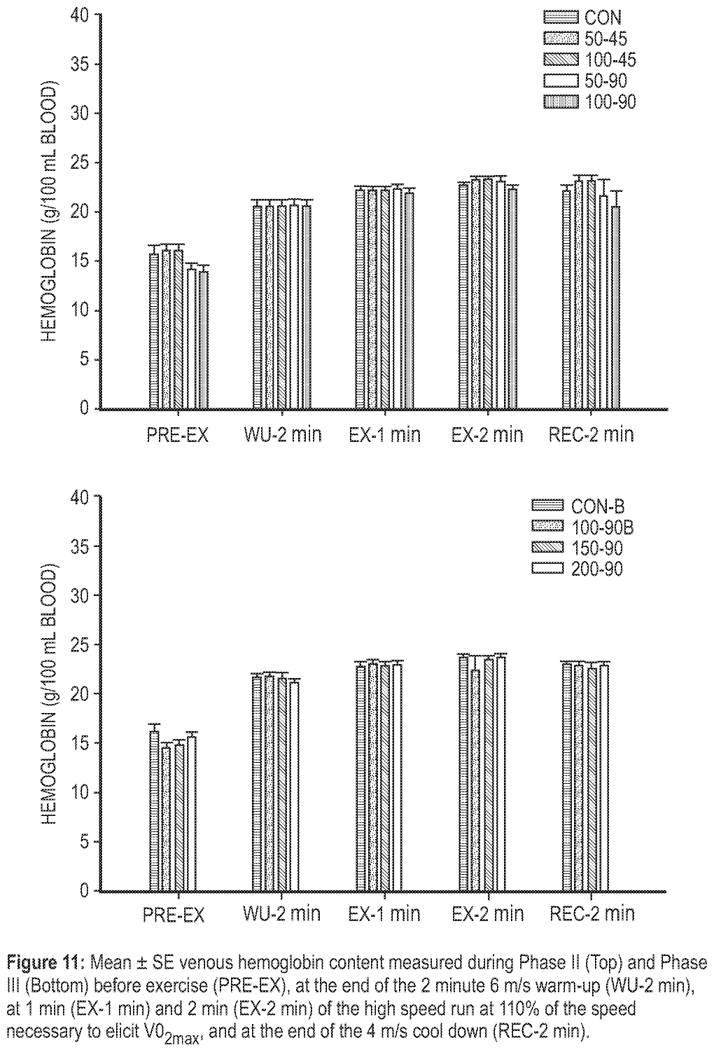

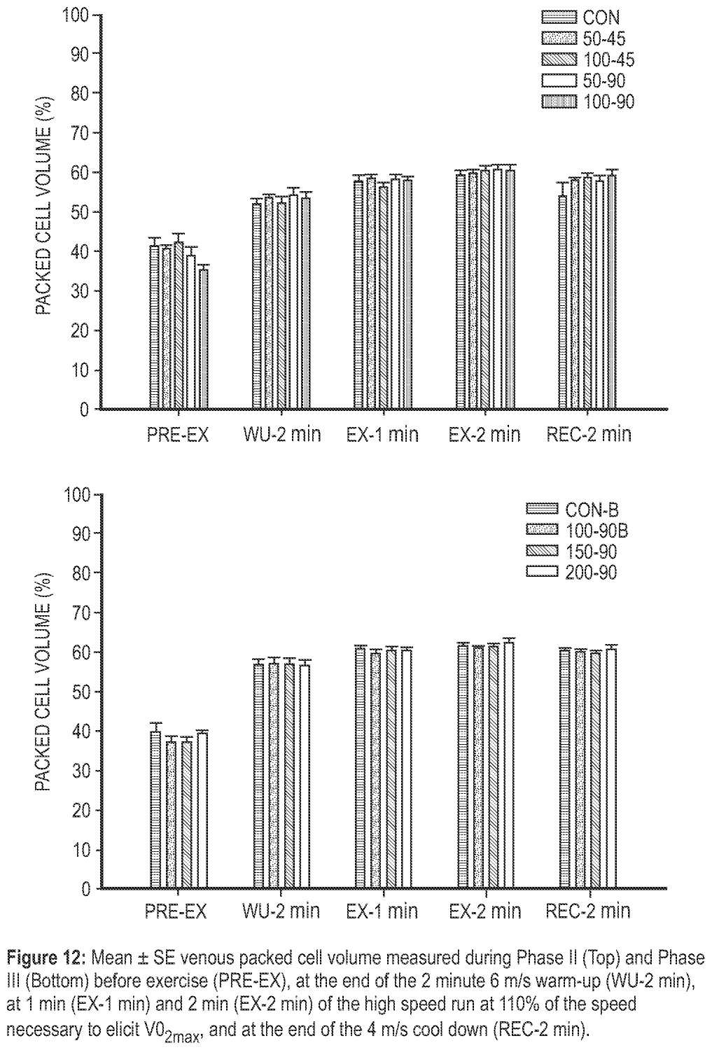

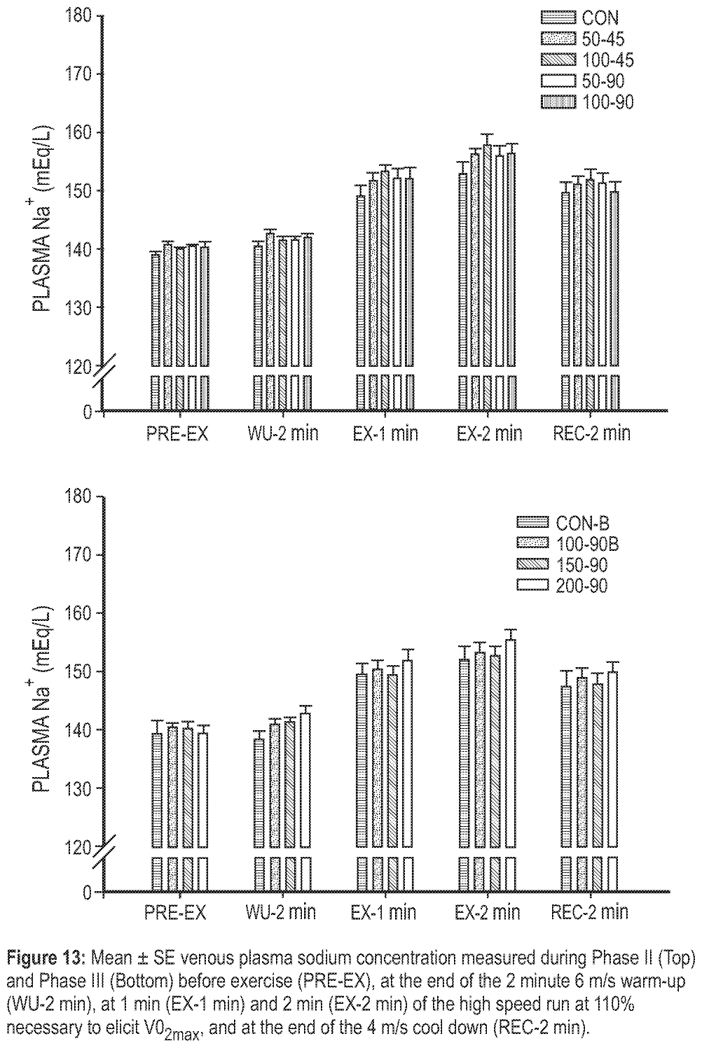

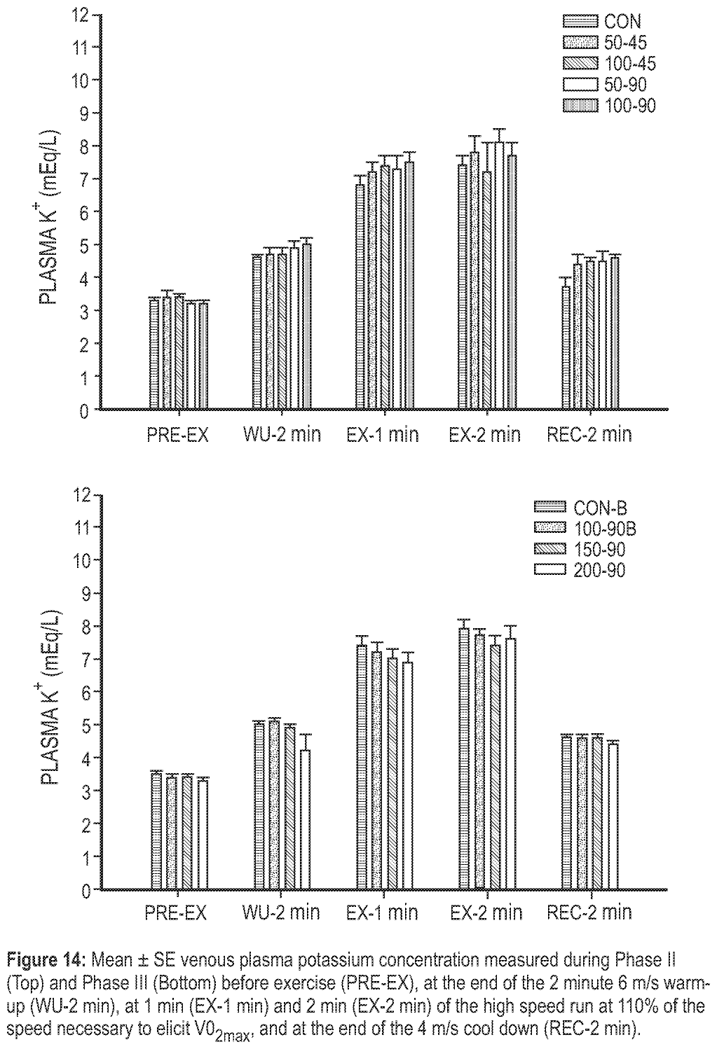

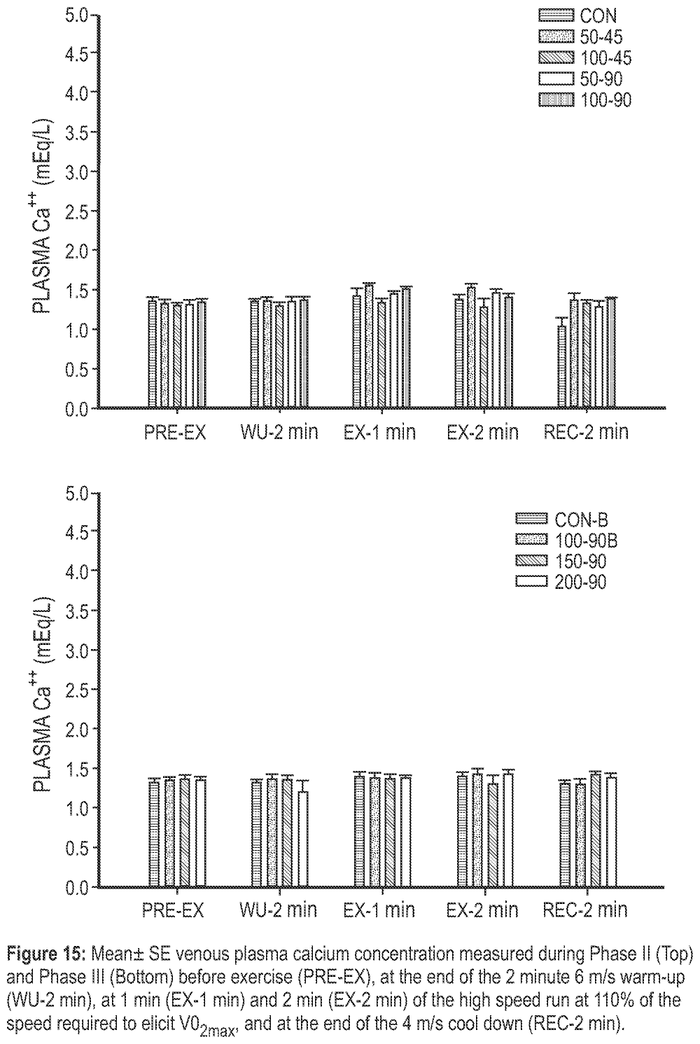

Blood Gases and Biomarkers: There was a significant effect of exercise as well as E-4021 on plasma glucose concentration during the SRTs (FIG. 5). There were significant effects of exercise on blood gas variables including decreases in PpaO₂ (FIG. 6), pH (FIG. 7), sO₂ (FIG. 8) and base ecf (FIG. 9). Exercise caused a significant increase in PpaCO₂ (FIG. 10), venous hemoglobin content (FIG. 11), packed cell volume (FIG. 12), sodium concentration (FIG. 13), and potassium concentration (FIG. 14). Lastly, there was no effect of exercise or E-4021 on plasma calcium concentration (FIG. 15).

P-values for ANOVA evaluating possible differences among treatments for the variables are shown in Table 5 below:

| | | | 1 min | 2 min | |
|---|---|---|---|---|---|
| | PRE | WARM-UP | exercise | exercise | RECOVERY |
| P_{PA} | NA | 0.029 | <0.001 | <0.001 | 0.515 |
| V_{O2} | NA | 0.983 | 0.260 | 0.399 | 0.957 |
| Lactate | NA | 0.962 | 0.951 | 0.927 | 0.876 |
| P_{PA}O₂ | NA | 0.426 | 0.866 | 0.434 | 0.982 |
| P_{PA}CO₂ | NA | 0.699 | 0.936 | 0.758 | 0.329 |
| PCV | NA | 0.393 | 0.095 | 0.616 | 0.222 |
| Na | 0.939 | 0.506 | 0.515 | 0.164 | 0.640 |
| K | 0.588 | 0.132 | 0.668 | 0.823 | 0.081 |
| pH | 0.472 | 0.776 | 0.601 | 0.954 | 0.379 |
| Ca | 0.974 | 0.731 | 0.214 | 0.560 | 0.228 |
| Glucose | 0.937 | 0.050 | 0.037 | 0.063 | 0.006 |
| BE | 0.732 | 0.474 | 0.978 | 0.956 | 0.999 |
| Hb | 0.092 | 0.427 | 0.710 | 0.820 | 0.889 |

In Table 5, P≤0.05 indicates significant differences between at least 2 of the treatments measured prior to exercise (PRE), at the end of the 2 minute 6 m/s warm-up, after 1 minute and 2 minutes high speed exercise at 110% of the speed required to elicit VO₂ₘₐₓ, and at the end of the 4 m/s cool down period (RECOVERY). Post-hoc tests (Dunnett and Tukey) were then conducted for those comparisons where P≤0.05.

Given these findings, a dose of 100 mg E-4021 (for an approximately 500 kg horse), 90 minutes prior to intense exercise, provides the greatest attenuation of increased exercise-related pulmonary vascular pressures, and thus potential attenuation of EIPH.

### Example 6: Evaluation of a New Formulation of a Novel Type 5 Phosphodiesterase Inhibitor for Strenuously Exercising Equine Using a Treadmill

This study was performed to evaluate a new formulation of a novel type-5 phosphodiesterase inhibitor, E-4021 to reduce pulmonary artery pressure (PAP) during treadmill exercise.

Conditioning was continued as per the SEP (standard exercise procedure). Randomized administration was performed using the one of the following treatments (one Tx per week): Treatment 1 (Con): Control; Treatment 2 (90 min): Dose: 100 mg PDE5 (which was E-4021, 15005), which contain meglumine using the formulation of Table 3, (˜200 ug/kg), SRT at 90 min post-injection.

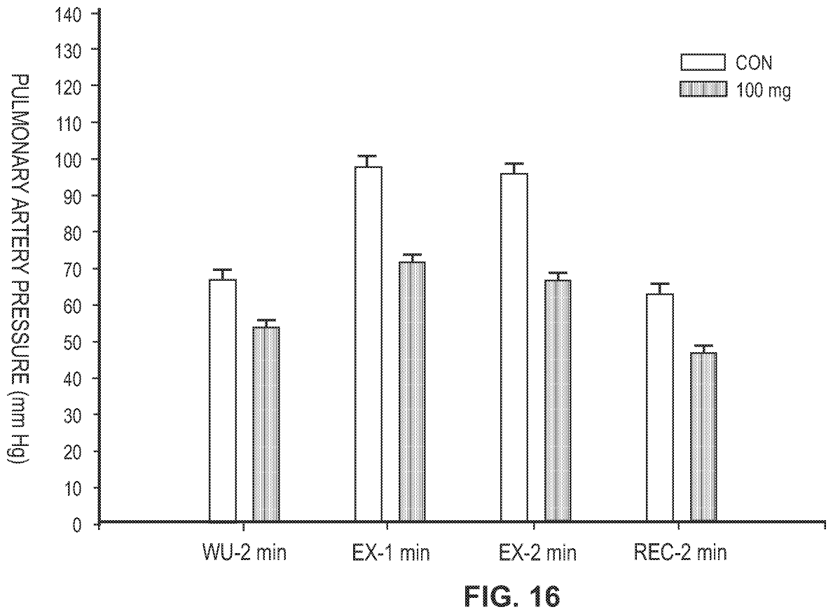

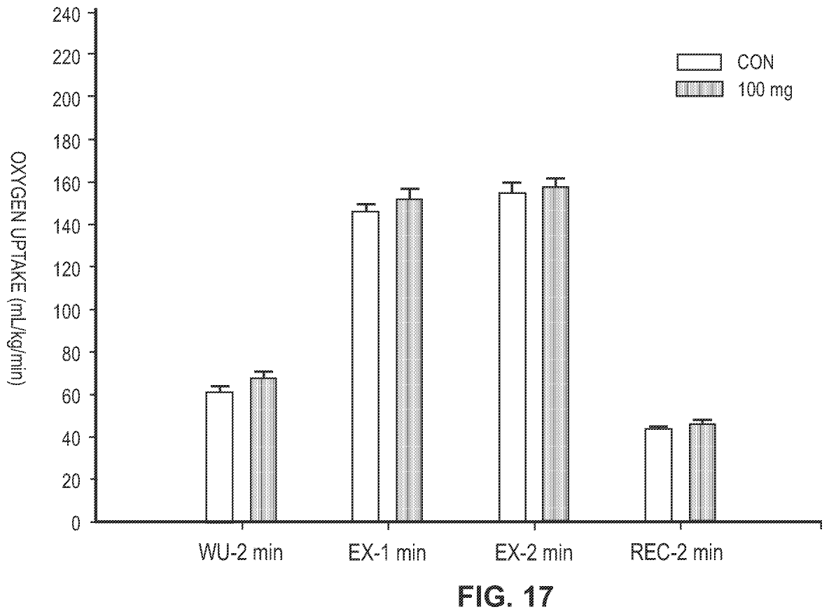

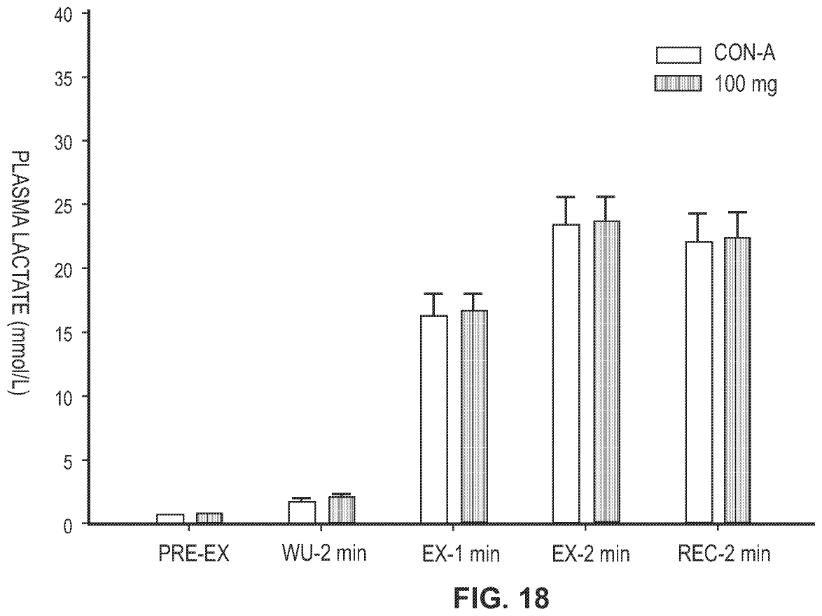

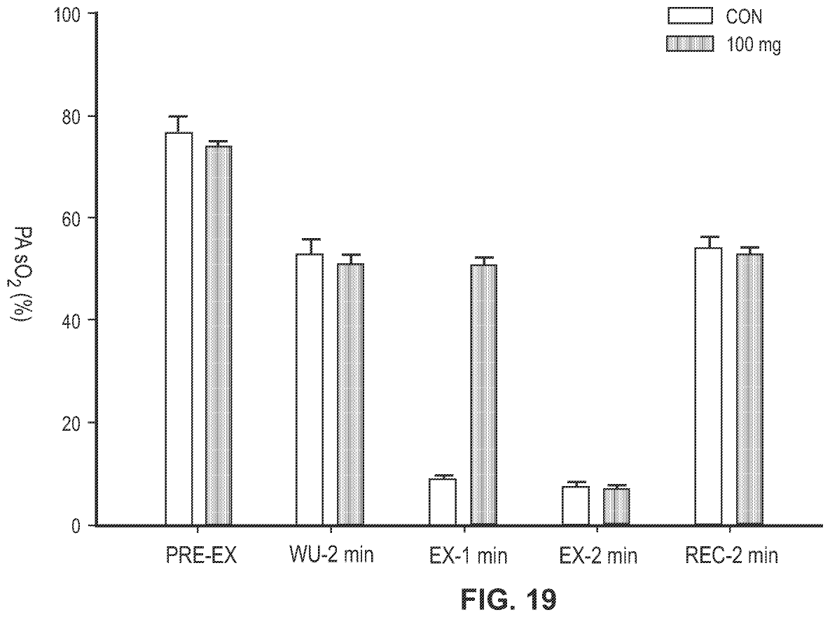

Data for the pulmonary artery pressure (FIG. 16) shows that the administration of a single dose of 100 mg PDE5 injection 90 minutes prior to SRT, reduces the pulmonary artery pressure in each case when compared to control. Other indices related to exercise capacity e.g., oxygen uptake (as shown in FIG. 17), plasma lactate (as shown in FIG. 18) and PAsO₂ (as shown in FIG. 19) were not significantly different following administration (Treatment 2) when compared to control (Treatment 1).

### Example 7: Duration of Effect of a Novel Type 5 Phosphodiesterase Inhibitor for Strenuously Exercising Equine Using a Treadmill

Conditioning was continued as per the SEP. Randomized administration was performed using the one of the following treatments (one Tx per week): Treatment 1 (Con): Control; Treatment 2 (90 min): Dose: 100 mg PDE5 (which was E-4021, 16006, which is the formulation of Table 3 containing meglumine), (˜200 ug/kg), SRT at 90 min post-injection; Treatment 3 (4 hrs.): Dose: 100 mg PDE5, (˜200 ug/kg), SRT at 4 hrs. post-injection; Treatment 4 (24 hrs.): Dose: 100 mg PDE5, (˜200 ug/kg), SRT at 24 hrs. post-injection; and Treatment 5 (48 hrs.): Dose: 100 mg PDE5, (˜200 ug/kg), SRT at 48 hrs. post-injection.

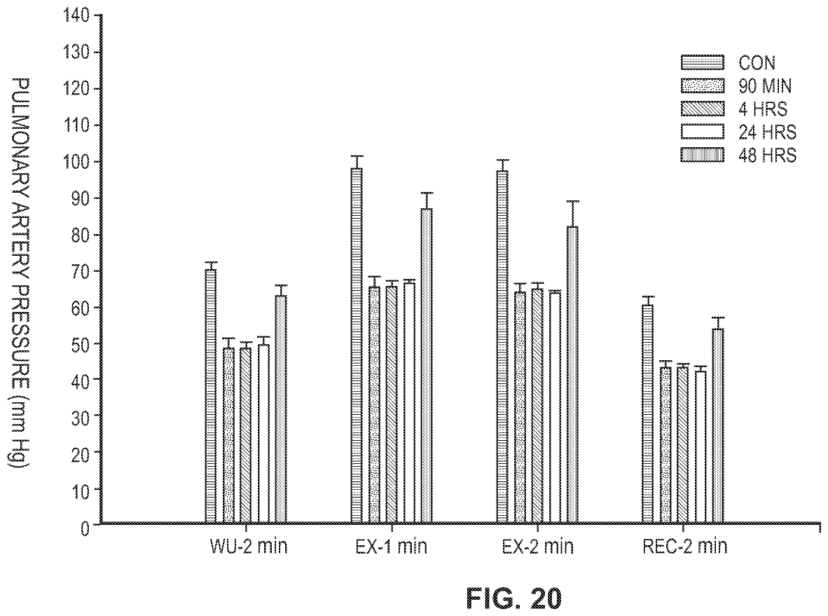

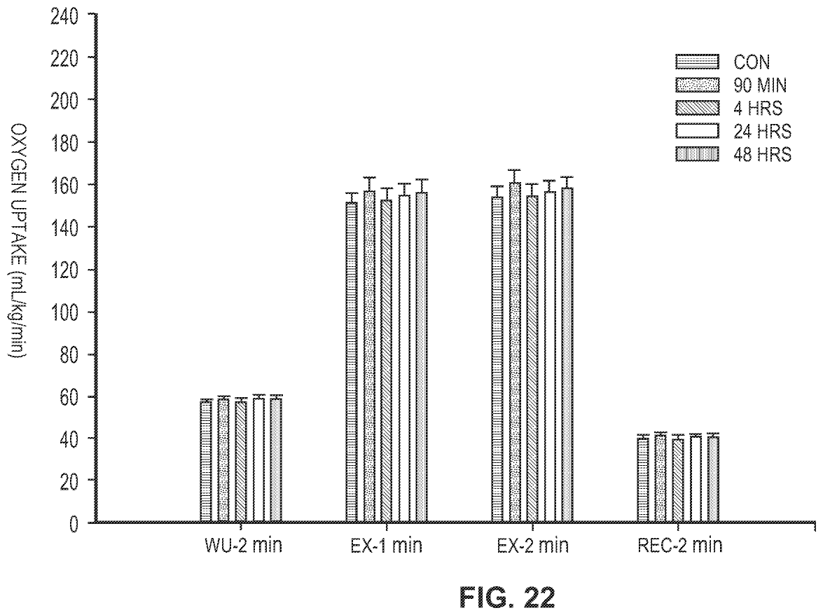

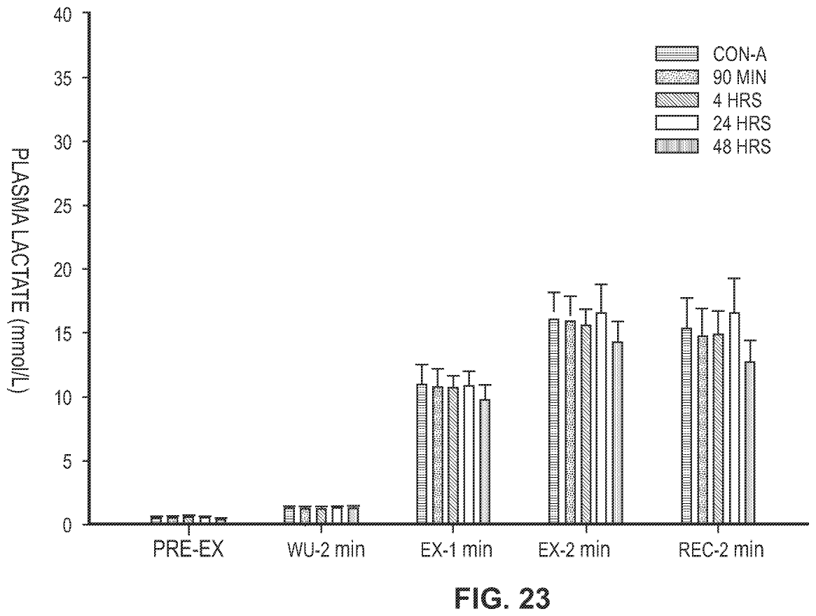

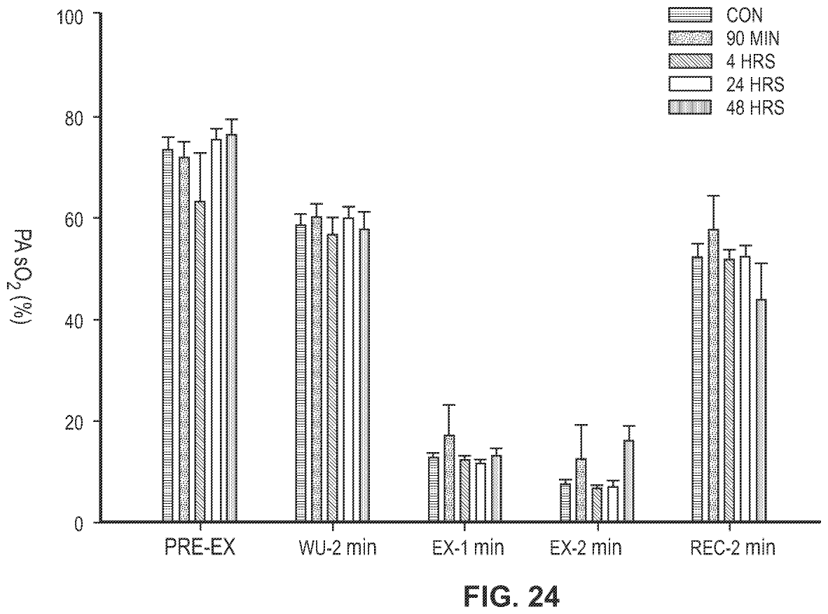

Data for the pulmonary artery pressure (FIG. 20) shows the duration of pulmonary artery pressure reduction effect of 100 mg PDE5 administration lasts at least 24 hours post-injection for SRT. FIG. 21 shows the data for the pulmonary artery pressure at the end of 2 minutes of intense treadmill exercise after 45 minutes, 90 minutes, 4 hours, 24 hours and 48 hours post dose with the PDE5 containing propylene glycol (PPG) and the PDE5 containing meglumine (MEG). Other indices are related to exercise capacity, e.g., oxygen uptake (as shown in FIG. 22) plasma lactate (as shown in FIG. 23) and PAsO₂ (as shown in FIG. 24), which were not significantly different following the administration of 100 mg PDE5 at different time points (90 min., 4 hrs., 24 hrs. and 48 hrs. post-injection) when compared to the control. This indicates that the PDE5 containing meglumine had an extended duration of action to lower PAP at 48 hours post dose and then the PAP began to elevate. It was concluded that the meglumine in the PDE5 extended the duration of action. This is believed to be due to the meglumine alkaline characteristics that stabilizes the formulation but also extends the duration of action.

### Example 8: Effects of a Type-5 Phosphodiesterase Inhibitor on Pulmonary Artery Pressure in Race Fit Horses

This study was performed to determine the optimal dose and timing of E-4021, which is PDE5 containing propylene glycol (PPG), to reduce pulmonary artery pressure (PAP) during treadmill exercise. The formulation used contained polypropylene glycol as indicated in Table 1. Eight (4 geldings, 4 mares) unfit Standardbreds (4-8 y, ˜490 kg) were conditioned for the entire trial. Speed and duration increased weekly until week 12-14, when three treadmill GXT were performed to document stable fitness (VO₂ₘₐₓ).

Two randomized crossover experiments then used simulated race tests (SRT) to determine dose and timing of IV administration of E-4021. Experiment-1: no drug (CON-A) or two doses (50 vs. 100 mg) and two time points (45 vs. 90 min). Experiment-2 (all 90 min): no drug (CON-B); 100 mg (100B); 150 mg; or 200 mg. The SRT used a 2-min warm-up; 2-min at 110% VO₂ₘₐₓ; 2-min recovery. PAP, ECG, VO₂, and VCO₂ were measured continuously and blood (3 mL) collected anaerobically at end of the warm-up, at 1 and 2 minutes at high speed, and at the end of recovery to measure PpaO₂, PpaCO₂, pH, sO₂, [Na+], [K+], [CA++], [lactate], [glucose], [hemoglobin], BE (Base Excess in extracellular fluid), and PCV. Analysis included repeated measures using ANOVA, Dunnett's and Tukey tests with P<0.05 considered significant.

The major finding was that the 100 mg dose administered 90 minutes before exercise resulted in the lowest PA pressure (P<0.05). There were no differences (P>0.05) in PAP during the 100 mg vs. 150 mg vs. 200 mg trials. E-4021 did not alter (P>0.05) markers of aerobic or anaerobic performance. The ˜30 mmHg lower PAP with 100 mg at 90 minutes before exercise represents a clinically significant effect.

### Example 9: Pharmacokinetic Properties of the Composition in Mature Horses

A pharmacokinetic (PK) study was conducted in six horses, namely three mares, two geldings and one stallion. The study measured plasma and urine concentrations of E-4021 (EIPHISOL®) after IV administration of 100 mg of E-4021 per horse. Plasma samples were collected pretreatment and then at 10, 20 and 30 minutes and 1, 1.5, 2, 2.5, 3, 3.5, 4, 4.5, 5, 5.5, 6, 8, 12, 17, 24 and 30 hours post administration. The mean maximum concentration (Cmax) and standard deviation (±SD) was 295±118 ng/mL. Mean Tmax was 0.195±0.020 hours. The mean elimination half-life (T½) was 4.42±2.91 hours. The mean area under the concentration-time curve extrapolated to infinity (AUC0-∞) was 217±83.5 hr*ng/mL. The mean volume of distribution (V) was 6.06±3.99 L/kg. The mean clearance (CL) was 1.17±0.690 L/hr/kg.

Urine was collected for assaying E-4021 during four intervals: (1) a pre-dose sample collected from approximately −24 hours through 0 hours, (2) a 0 hour to 12 hour post-dose sample, (3) a 12 hour to 24 hour post-dose sample, and, (4) a 24 hour to 36 hour-post dose sample. There was wide variability in the concentration of E-4021 measured in urine, with maximum concentration ranging from 130-974 ng/mL. The maximum concentration for all six horses was measured during the 12-hour post-dose collection. E-4021 concentration in urine remained above the quantitation limit (3 ng/mL) for four of the six horses at 36 hours post-dose.

### Example 10: Animal Safety

A twenty-four week (6-month) target animal safety (TAS) study was conducted to evaluate the safety of E-4021 (EIPHISOL®) in mature, healthy horses. The study was designed with 4 treatment groups of eight horses (two geldings, two stallions and four mares) in each group. Treatment groups included a Control (Group I: Isotonic saline at a volume equivalent to the largest volume given in the 5× (Group IV); 1× (Group II: 0.125 mg E4021 per pound body weight); 3× (Group III: 0.375 mg E4021 per pound body weight; and 5× (Group IV: 0.625 mg E4021 per pound body weight). Treatment groups were dosed intravenously alternating between the left and right jugular vein once every 7 days for 25 doses.

Breeds represented in this study included Thoroughbred, Quarter Horse, Paint, Arabian, and Grade. The overall age ranged from 3 to 17 years, with a mean of 7.6±4.49 years. The majority of the horses were Thoroughbred (59.4%).

Microscopic findings associated with E-4021 at the injection site(s) consisted of minimal fibrosis of the dermis or vein, and minimal degeneration and necrosis of the underlying skeletal muscle. These findings were attributed to minor trauma associated with the injection procedure. There were no macroscopic findings limited to the injection site.

All horses (except two of the 32) appeared healthy throughout the course of the study. Following the initiation of dosing, two horses succumbed to abdominal colic (horse 62 in Group II (1×) on Study Day 51 and horse 117 in Group I (Control) on Study Day 54). The adverse event of abdominal colic did not appear to be associated with test article administration.

Physical Examinations and Post-Dose Observations: Post-dose observations were conducted after every treatment to detect any acute abnormalities at 10-, 30-, and 60-minute intervals. There were no major abnormal physical examination findings noted for the horses that were treatment-related at 1× and 3×. Group IV (5×) appeared to be overrepresented on the physical examinations' findings at and immediately after dosing as it appeared that at the high dose, E-4021 caused transient locomotion (ataxia/dragging toes), or neurodepressant type effects (behavioral/sedation). As all groups (including the controls) recorded this finding (Table I), it is presumed that a factor other than the test article was responsible. Likewise, these findings did not appear to occur in a dose related fashion, thus were not considered treatment related.

TABLE 8
Visual Findings Related to Ataxia/Dragging Toes (Locomotion)
or Behavioral/Sedation (Neurodepressant) Effects Counted Once
per Horse per Group for each Physical Examination (PE) Day

| Dose Group | Control | 1X | 3X | 5X |
|---|---|---|---|---|
| PE Day 0 | 3 | 6 | 2 | 6 |
| PE Day 28 | 4 | 4 | 0 | 4 |
| PE Day 56 | 2 | 2 | 1 | 3 |
| PE Day 84 | 1 | 1 | 0 | 4 |
| PE Day 112 | 0 | 1 | 0 | 2 |
| PE Day 140 | 0 | 1 | 1 | 2 |
| PE Day 168 | 0 | 1 | 0 | 2 |

In general, the frequency of these physical examination findings appeared to decrease by 60 minutes post dose.

Urine and Fecal Analysis: There did not appear to be any indication of abnormal urinalysis findings or changes noted in urinalysis parameters.

There were no indications of abnormal fecal analysis findings except for the following: Two horses in Group III (3×) had abnormal intestinal mucosa at Day 83, whereas the other groups had normal intestinal mucosa. One horse in Group II (1×) and 2 horses in Group III (3×) tested positive for parasites at Day −14 (all horses were dewormed following this finding). All horses tested negative for parasites at Day 83.

Clinical Chemistry Parameters: The treatment group-by-time-interaction was statistically significant for blood urea nitrogen (BUN) and glucose. For BUN, horses in Group IV (5×) had significantly lower BUN levels on Days 28, 42, 84, and 112 than in Group I (Control). Blood urea nitrogen levels on Day 140 for Group II (1×) and Day 154 for Group III (3×) were significantly higher than in Group I (Control). For glucose, horses in the Group II (1×) had significantly lower glucose levels on Days 28, 42, 56, 70, and 140 than in Group I (Control). Glucose values on Days 28, 84, 140, and 168 were significantly lower in Group IV (5×) than in the Group I (Control).

The treatment group-by-sex-interaction was statistically significant for albumin, globulin, magnesium, and sorbitol dehydrogenase. For albumin, females in Group II (1×) and Group III (3×) had significantly lower albumin values than in Group I (Control). Males in Group II (1×) and Group III (3×) had significantly higher albumin values than in Group I (Control). For globulin, females in Group II (1×) had significantly higher globulin values than in the Group I (Control). Males in Group III (3×) and Group IV (5×) had significantly lower globulin values than in the Group I (Control). For magnesium, males in Group IV (5×) had significantly lower magnesium values than in Group I (Control). In females, there was no significant difference between treatment groups and control. For sorbitol dehydrogenase, females in Group II (1×) and Group IV (5×) had significantly higher sorbitol dehydrogenase values than in Group I (Control). Males in Group II (1×) and Group III (3×) had significantly higher sorbitol dehydrogenase values than in Group I (Control).

The main effect of the treatment group was statistically significant for direct bilirubin. Overall, Group II (1×) had significantly lower direct bilirubin values than in in Group I (Control).

Hematology and Coagulation: The treatment group-by-time-interaction was statistically significant for activated partial thromboplastin time (APTT), mean corpuscular hemoglobin concentration (MCHC), mean corpuscular hemoglobin (MCH), and leukocytes. For APTT, horses in Group II (1×) had significantly higher APTT levels on Days 56, 140, and 168 than in Group 1 (Control). Horses in Group III (3×) had significantly higher APTT levels on Days 28, 56, 98, 140, and 168 than in Group I (Control). Horses in Group IV (5×) had significantly higher APTT levels on Days 56, 70, and 126 and significantly lower levels at Day 84 than horses in Group I (Control). For MCHC, horses in Group II (1×) had significantly higher MCHC levels on Days 14, 70, and 154 than horses in Group I (Control). Horses in Group III (3×) had significantly lower MCHC levels on Day 0 than in Group I (Control). Horses in Group IV (5×) had significantly higher MCHC levels on Days 98, 112, and 154 than horses in Group I (Control). For MCH, horses in Group III (3×) had significantly higher MCH levels on Day 56 than in Group I (Control). There were no significant differences between Group II (1×) and Group IV (5×) and Group I (Control). For leukocytes, horses in Group II (1×) had significantly lower leukocyte levels on Days 14 and 154 than horses in Group I (Control). Horses in Group III (3×) had significantly higher leukocyte levels on Days 84, 98, 112, 140, and 168 than horses in Group I (Control). There were no significant differences between Group IV (5×) and Group I (Control).

The treatment group-by-sex-interaction was statistically significant for leukocytes, lymphocytes/leukocytes, and neutrophils/leukocytes. For leukocytes, female horses in Group III (3×) and Group IV (5×) had significantly higher leukocyte values than female horses in Group I (Control). Males in Group II (1×) had significantly lower leukocyte values than male horses in Group I (Control). For lymphocytes/leukocytes, males in Group III (3×) and Group IV (5×) had significantly higher lymphocytes/leukocytes values than males in Group I (Control). In females, there were no significant differences between treatment groups and control. For neutrophils/leukocytes, males in Group IV (5×) had significantly lower neutrophils/leukocytes values than in Group I (Control). In females, there were no significant differences between treatment groups and control.

The main effect of the treatment group was statistically significant for erythrocytes, fibrinogen, hematocrit, and hemoglobin. Overall, Group II (1×) had significantly lower erythrocytes, hematocrit, and hemoglobin values than in Group I (Control). Overall, Group III (3×) had significantly lower fibrinogen values than in Group I (Control).

Bone Marrow Smears: A low incidence of decreased megakaryocytes was noted in control and test-article treated horses without a dose relationship or correlation with decreased platelet counts on Day 168. Low cellularity and/or hemodiluted specimens were attributed to sampling artifacts. All other differences in bone marrow smears were consistent with normal biological variation.

Macroscopic and Histopathological Evaluations: Some macroscopic observations pre-dated the initiation of dosing (fetlock cavity [microscopic hemorrhage and fibrosis] and skin mass [not examined microscopically]), were identified only in control horses (adrenal gland mass [microscopic cortical adenoma] and heart discoloration [microscopic endocardial mineralization]), or were attributed to euthanasia artifact (injection site discoloration [microscopic hemorrhage]).

Other macroscopic observations were identified at comparable incidence across dose groups, including the control group (stomach, non-glandular ulcers [microscopic erosions in most], and lung and liver nodules [microscopic abscesses], sometimes associated with liver adhesions [fibrosis]).

Still other macroscopic observations were identified only sporadically (thyroid gland mass [microscopic follicular cell nodular hyperplasia], ovarian cyst [microscopic cyst], stomach mass [microscopic non-glandular papilloma], stomach adhesion [microscopic granulomatous inflammation of the mesentery/serosa], mediastinal lymph node discoloration [microscopic granulomatous inflammation], duodenal dilation [no microscopic correlate], uterine mass [no microscopic correlate], and abdominal (cavity) mass [microscopic fat necrosis].

All macroscopic observations identified at the end of dosing were attributed to spontaneous background alterations associated with previous trauma, previous infectious diseases, and/or degenerative/aging changes or were attributed to euthanasia artifact.

Microscopic Findings: Testes: Minimal seminiferous tubule degeneration, often with minimal or mild, unilateral or bilateral mononuclear cell infiltration and/or minimal or mild, multifocal to diffuse Leydig cell pigment accumulation were identified in the testes of horses that were administered E-4021, but not the testes of the single control male (stallion 116) that survived to study termination. The testicular changes in horses that were administered E-4021 did not show evidence of a dose response to the test article and were most consistent with spontaneous background findings.

The mandibular salivary glands were not available for microscopic examination for any of the females that were given E-4021 or any males given 5× E-4021. In the limited number of mandibular salivary glands examined for males given 1× and 3× E-4021, there were no microscopic findings identified that were related to the test article administration.

Organ to Body Weight Ratio: The Group-by-Sex interaction was statistically significant for liver to body weight (%) and liver to brain weight (%). In the pairwise comparisons (LSMEANS) of liver weight ratios, males given the 3× dose of E-4021 had statistically higher mean liver to body weight ratio (%) (p=0.064) and liver to brain weight ratio (%) (p=0.033) compared to control males. The difference was not present in females and the magnitude of the difference in males was small. There was no evidence of a dose response in the males or correlative macroscopic or microscopic findings.

The references below are herein incorporated by reference.

### REFERENCES

Birks, E. K., Jones, J. H., Vandervort, L. J., Priest, A. K., and Berry, J. D. 1991. Plasma Lactate Kinetics during Exercise. Equine Exercise Physiology 3:179-187.
Kearns, C. F. and K. H. McKeever Clenbuterol diminishes aerobic performance in horses Medicine and Science in Sport and Exercise, 34:1976-1985, 2002.
Liburt, N. R., K. H. McKeever, J. M. Streltsova, W. C. Franke, M. E. Gordon, H. C. Manso, Filho, D. W. Horohov, R. T. Rosen, C. T. Ho, A. P. Singh, N. Vorsa. Effects of cranberry and ginger on the physiological response to exercise and markers of inflammation following acute exercise in horses. Comparative Exercise Physiology. 6:157-169, 2009.
McKeever, J. M., K. H. McKeever, J. Alberici, M. E. Gordon, and H. C. Manso, Filho Effect of Gastrogard on markers of performance in Standardbred horses. Equine Veterinary Journal Suppl. 36:668-671, 2006.
McKeever, K. H., J. M. Agans, S. Geiser, P. Lorimer, and G. A. Maylin. Low dose exogenous erythropoietin elicits an ergogenic effect in Standardbred horses. Equine Veterinary Journal Suppl. 36:233-238, 2006.
Seeherman, H. J., and Morris, E. A. Methodology and repeatability of a standardized treadmill exercise test for clinical evaluation of fitness in horses. Equine Vet. J. Suppl. 9:20-25, 1990.
Streltsova, J. M., K. H. McKeever, N. R. Liburt, H. C. Manso, M. E. Gordon, D. Horohov, R. Rosen, W. Franke. Effect of orange peel and black tea extracts on markers of performance and cytokine markers of inflammation in horses. Equine and Comparative Exercise Physiology 3:121-130, 2006.
Rose, R. J., Hodgson, D. R., Kelso, T. B., McCutcheon, L. J., Reid, T-A, Bayly, W. M., and Collnick, P. D. Maximum 02 uptake, 02 debt and deficit, and muscle metabolites in Thoroughbred horses. J. Applied Physiology 64(2):781-788, 1988

It should be understood that the forgoing relates to exemplary embodiments of the disclosure and that modifications may be made without departing from the spirit and scope of the disclosure as set forth in the following claims.

1. A method of preventing or treating exercise-induced pulmonary hemorrhage in a mammal in need thereof, the method comprising administering a composition comprising a type V phosphodiesterase inhibitor and an amino sugar to the mammal.

2. The method of claim 1, wherein the composition is administered systemically or locally.

3. The method of claim 1, wherein the composition is administered by a route of administration comprising intravenous, intramuscular, subcutaneous, intraperitoneal, intraarterial, inhalation, topical, or intradermal routes.

4. The method of claim 3, wherein the composition is administered intravenously, and the mammal is a horse.

5. The method of claim 1, wherein the type V phosphodiesterase inhibitor comprises one of sildenafil, avanafil, iodenafil, mirodenafil, tadalafil, vardenafil, udenafil, zaprinast, icariin, benzamidenafil, dasantafil, dipyridamole, tadalafil, E-4021, which is sodium 1-[6-chloro-4-(3,4-methylenedioxybenzyl)-aminoquinazolin-2-yl]piperidine-4-carboxylate sesquihydrate, which is E4010 4-(3-chloro-4-methoxybenzyl)amino-1-(4-hydroxypiperidino)-6-phthalazinecarbonitrile monohydrochloride, DMPPO (1,3-dimethyl-6-(2-propoxy-5-methanesulfonyl-amidophenyl)pyrazol[3,4d]-pyrimidin-4-(5H)-one) or a combination thereof.

6. The method of claim 5, wherein the type V phosphodiesterase inhibitor comprises E-4021, which is sodium 1-[6-chloro-4-(3,4-methylenedioxybenzyl)-aminoquinazolin-2-yl]piperidine-4-carboxylate sesquihydrate.

7. The method of claim 1, wherein the type V phosphodiesterase inhibitor is administered at a dosage of from about 5 μg/kg to about 500 μg/kg to the mammal.

8. The method of claim 1, wherein the mammal is a horse and the composition comprises E-4021, which is sodium 1-[6-chloro-4-(3,4-methylenedioxybenzyl)-aminoquinazolin-2-yl]piperidine-4-carboxylate sesquihydrate administered by injection at a dose of 50 mg, 100 mg, 150 mg, or 200 mg from about 30 minutes to about 7 days prior to strenuous exercise.

9. The method of claim 1, wherein the composition comprises from about 0.05% w/w or w/v to about 40% w/w or w/v of the amino sugar, which is meglumine based on a total weight of the composition.

10. The method of claim 1, wherein the composition comprises from about 10% w/w or w/v to about 60% w/w or w/v of alcohol based on a total weight of the composition.

11. The method of claim 1, wherein the composition comprises E-4021, which is sodium 1-[6-chloro-4-(3,4-methylenedioxybenzyl)-aminoquinazolin-2-yl]piperidine-4-carboxylate sesquihydrate at a dose of 50 mg, 100 mg, 150 mg, or 200 mg, the amino sugar which is meglumine in an amount of 25 mg, dehydrated alcohol in an amount of 3.94 g, and water for injection.

12. The method of claim 1, wherein the composition reduces pulmonary arterial pressure to about 90 mm Hg or less during an exercise event that produces pulmonary vascular pressure greater than 90 mm Hg.

13.-49. (canceled)

50. An aqueous composition for preventing or treating exercise-induced pulmonary hemorrhage in a mammal, the aqueous composition comprising a type V phosphodiesterase inhibitor, an organic base or amino sugar and water.

51. The aqueous composition of claim 50, wherein the organic base or amino sugar comprises meglumine.

52. (canceled)

53. The aqueous composition of claim 50, wherein the type V phosphodiesterase inhibitor reduces pulmonary arterial pressure to about 90 mm Hg or less, about 30 minutes, 45 minutes, 90 minutes, 4 hours, 24 hours, 48 hours, 72 hours to about 96 hours after the type V phosphodiesterase inhibitor is administered to the mammal during an exercise event that produces pulmonary vascular pressure greater than 90 mm Hg.

54. An aqueous composition for preventing or treating elevated pulmonary vascular pressure in a mammal, the aqueous composition comprising a type V phosphodiesterase inhibitor, an organic base or amino sugar and water.

55. The aqueous composition of claim 54, wherein the organic base or amino sugar comprises meglumine.

56. The aqueous composition of claim 54, wherein the organic base or amino sugar comprises meglumine, L-arginine, triethylamine, diethylamine, ethanolamine, diethanolamine, triethanolamine, ethylenediamine, or a combination thereof.

57. (canceled)

58. The aqueous composition of claim 54, wherein the type V phosphodiesterase inhibitor reduces pulmonary arterial pressure to about 90 mm Hg or less, about 30 minutes, 45 minutes, 90 minutes, 4 hours, 24 hours, 48 hours, 72 hours to about 96 hours after the type V phosphodiesterase inhibitor is administered to the mammal during an exercise event that produces pulmonary vascular pressure greater than 90 mm Hg.

59. The aqueous composition of claim 54, wherein the composition has a pH between about 7.1 to about 12.

60.-108. (canceled)
